(19) United States
(12) Patent Application Publication
(10) Pub. No.: US 2006/0127880 A1
(43) Pub. Date: Jun. 15, 2006

(54) Computerized image capture of structures of interest within a tissue sample

(76) Inventors: Walter Harris, Bremerton, WA (US); Phillip Freund, Bremerton, WA (US); Robert Cascisa, Poulsbo, WA (US); Glenna C. Burmer, Seattle, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/195,255

(22) Filed: Aug. 1, 2005

Related U.S. Application Data
(63) Continuation of application No. 11/014,196, filed on Dec. 15, 2004, now abandoned.

(51) Int. Cl. C12Q 1/00; G06F 19/00; G06K 9/00

(52) U.S. Cl. 435/4; 702/19; 382/128

(57) ABSTRACT

A computerized method of automatically capturing an image of a structure of interest in a tissue sample. The computer memory receives a first pixel data set representing an image of the tissue sample at a low resolution and an identification of a tissue type of the tissue sample, and selecting at least one structure-identification algorithm responsive to the tissue type from a plurality of structure-identification algorithms, at least two of which are responsive to different tissue types. Each structure-identification algorithm correlates at least one cellular pattern in a given tissue type with a presence of a structure of interest for the given tissue type. The method also includes applying the selected structure-identification algorithm to the first pixel data set to determine a presence of the structure of interest in the tissue sample, and capturing a second pixel data set at a high resolution.

### PRIORITY

This application claims priority of U.S. provisional patent application No. 60/389,859 entitled VIRTUAL HISTOLOGY ALGORITHM DEVELOPMENT filed Jun. 18, 2002. This and all other references set forth herein are incorporated herein by reference in their entirety and for all teachings and disclosures, regardless of where the references may appear in this application.

### BACKGROUND

Medical research and treatment require rapid and accurate identification of tissue types, tissue structures, tissue substructures, and cell types. The identification is used to understand the human genome, interaction between drugs and tissue, and treat disease. Pathologists historically have examined individual tissue samples through microscopes to locate structures of interest within each tissue sample, and made identification decisions based in part upon features of the located structures of interest. However, pathologists are not able to handle the present volume of tissue samples requiring identification. Furthermore, because pathologists are human, the current process relying on time-consuming visual tissue analysis is inherently slow, expensive, and suffers from normal human variations and inconsistencies.

Adding to the volume of tissue samples requiring identification is a recent innovation using tissue microarrays for high-throughput screening and analysis of hundreds of tissue specimens on a single microscope slide. Tissue microarrays provide benefits over traditional methods that involve processing and staining hundreds of microscope slides because a large number of specimens can be accommodated on one master microscope slide. This approach markedly reduces time, expense, and experimental error. To realize the full potential of tissue microarrays in high-throughput screening and analysis, a fully automated system is needed that can match or even surpass the performance of a pathologist working at the microscope. Existing systems for tissue identification require high-magnification or high-resolution images of the entire tissue sample before they can provide meaningful output. The requirement for a high-resolution image slows capture of the image, requires significant memory and storage, and slows the identification process. An advantageous element for a fully automated system is a device and method for capturing high-resolution images of each tissue sample limited to structures of interest portions of the tissue sample. Another advantageous element for a fully automated system is an ability to work without requiring the use of special stains or specific antibody markers, which limit versatility and speed of the throughput.

In view of the foregoing, there is a need for a new and improved device and method for automated identification of structures of interest within tissue samples and for capturing high-resolution images that are substantially limited to those structures. The present invention is directed to a device, system, and method.

### SUMMARY

An embodiment of the present invention provides a computerized device and method of automatically capturing an image of a structure of interest in a tissue sample. The method includes receiving into a computer memory a first pixel data set representing an image of the tissue sample at a low resolution and an identification of a tissue type of the tissue sample, and selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type. The method also includes applying the selected at least one structure-identification algorithm to the first pixel data set to determine a presence of the structure of interest in the tissue sample, and capturing a second pixel data set at a higher resolution.

This computerized device and method provides automated capture of high-resolution images of structures of interest. The high-resolution images may be further used in an automated system to understand the human genome, interaction between drugs and tissue, and treat disease, or may be used without further processing.

These and various other features as well as advantages of the present invention will be apparent from a reading of the following detailed discussion and a review of the associated drawings.

### BRIEF DESCRIPTION OF THE DRAWINGS

The invention, together with further objects and advantages thereof, may best be understood by making reference to the following discussion taken in conjunction with the accompanying drawings, in the several figures of which like referenced numerals identify like elements, and wherein:

FIG. 1A illustrates a robotic pathology microscope having a lens focused on a tissue-sample of a tissue microarray mounted on a microscope slide, according to an embodiment of the invention;

FIG. 1B illustrates an auxiliary digital image of a tissue microarray that includes an array level digital image of each tissue sample in the tissue microarray, according to an embodiment of the invention;

FIG. 1C illustrates a digital tissue sample image of the tissue sample acquired by the robotic microscope at a first resolution, according to an embodiment of the invention;

FIG. 1D illustrates a computerized image capture system providing the digital tissue image to a computing device in a form of a first pixel data set at a first resolution, according to an embodiment of the invention;

FIG. 2 is a class diagram illustrating several object class families in an image capture application that automatically captures an image of a structure of interest in a tissue sample, according to an embodiment of the invention;

FIG. 3 is a diagram illustrating a logical flow of a computerized method of automatically capturing an image of a structure of interest in a tissue sample, according to an embodiment of the invention; and

FIGS. 4A-G illustrate steps in detecting a structure of interest in a kidney cortex, according to an embodiment of the invention.

### DETAILED DESCRIPTION

In the following detailed discussion of exemplary embodiments of the invention, reference is made to the accompanying drawings, which form a part hereof. The detailed discussion and the drawings illustrate specific exemplary embodiments by which the invention may be practiced. It is understood that other embodiments may be utilized, and other changes may be made, without departing from the spirit or scope of the present invention. The following detailed discussion is therefore not to be taken in a limiting sense, and the scope of the present invention is defined by the appended claims. A reference to the singular includes a reference to the plural unless otherwise stated or inconsistent with the disclosure herein.

Some portions of the discussions that follow are presented in terms of algorithms and symbolic representations of operations on data bits within a computing device. An algorithm is here, and generally is conceived to, be a self-consistent sequence of steps leading to a desired result. These steps require physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing terms such as “processing” or “computing” or “calculating” or “determining” or “displaying” or the like, refer to actions and processes of an electronic computing device, such as a computer system or similar device, that manipulates and transforms data represented as physical (electronic) quantities within the computer system's registers and memories into other data similarly represented as physical quantities within the computer system memories or registers or other such information storage, transmission, or display devices.

The process used by histologists and pathologists includes visually examining tissue samples containing cells having a fixed relationship to each other and identifying patterns that occur within the tissue. Different tissue types have different structures and substructures of interest to an examiner (hereafter collectively “structures of interest”), a structure of interest typically having a distinctive pattern involving constituents within a cell (intracellular), cells of a single type, or involving constituents of multiple cells, groups of cells, and/or multiple cell types (intercellular).

The distinctive cellular patterns are used to identify tissue types, tissue structures, tissue substructures, and cell types within a tissue. Recognition of these characteristics need not require the identification of individual nuclei, cells, or cell types within the sample, although identification can be aided by use of such methods. Individual cell types within a tissue sample can be identified from their relationships with each other across many cells, from their relationships with cells of other types, from the appearance of their nuclei, or other intracellular components.

Tissues contain specific cell types that exhibit characteristic morphological features, functions, and/or arrangements with other cells by virtue of their genetic programming. Normal tissues contain particular cell types in particular numbers or ratios, with a predictable spatial relationship relative to one another. These features tend to be within a fairly narrow range within the same normal tissues between different individuals. In addition to the cell types that provide a particular organ or tissue with the ability to serve its unique functions (for example, the epithelial or parenchymal cells), normal tissues also have cells that perform functions that are common across organs, such as blood vessels that contain hematologic cells, nerves that contain neurons and Schwann cells, structural cells such as fibroblasts (stromal cells) outside the central nervous system, some inflammatory cells, and cells that provide the ability for motion or contraction of an organ (e.g., smooth muscle). These cells also form patterns that tend to be reproduced within a fairly narrow range between different individuals for a particular organ or tissue, etc.

Histologists and pathologists typically examine specific structures of interest within each tissue type because that structure is most likely to contain any abnormal states within a tissue sample. A structure of interest typically includes the cell types that provide a particular organ or tissue with its unique function. A structure of interest can also include portions of a tissue that are most likely to be targets for treatment of drugs, and portions that will be examined for patterns of gene expression. Different tissue types generally have different structures of interest. However, a structure of interest may be any structure or substructure of tissue that is of interest to an examiner.

As used in this document, reference to “cells in a fixed relationship” generally means cells that are normally in a fixed relationship in the organism, such as a tissue mass. Cells that are aggregated in response to a stimulus are not considered to be in a fixed relationship, such as clotted blood or smeared tissue.

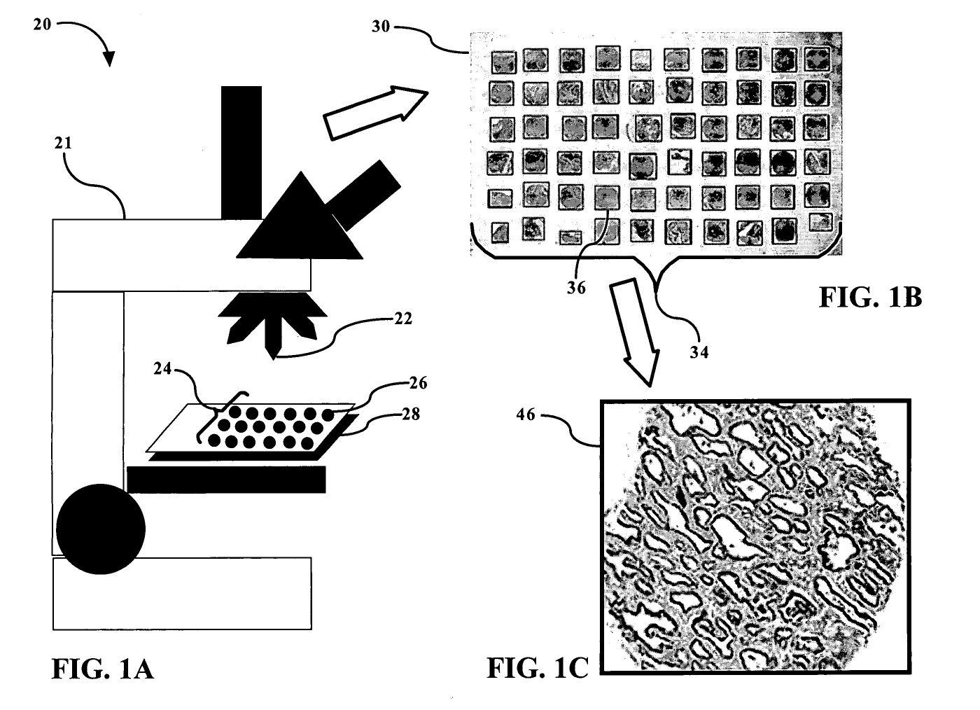

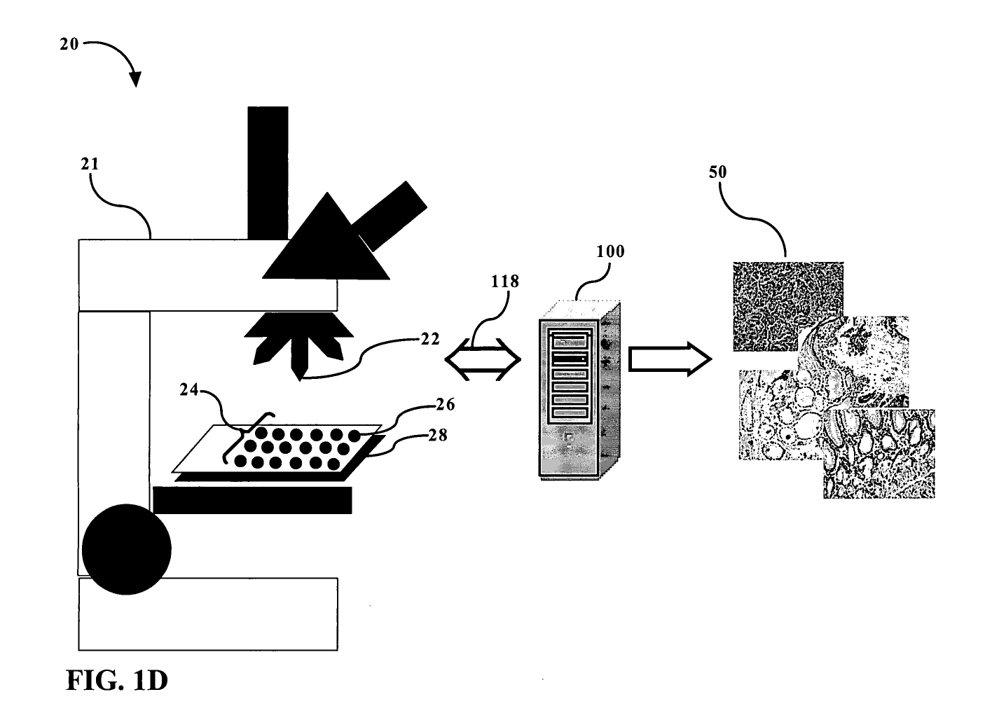

FIGS. 1A-D illustrate an image capture system 20 capturing a first pixel data set at a first resolution representing an image of a tissue sample of a tissue microarray, and providing the first pixel data set to a computing device 100, according to an embodiment of the invention. FIG. 1A illustrates a robotic pathology microscope 21 having a lens 22 focused on a tissue-sample section 26 of a tissue microarray 24 mounted on a microscope slide 28. The robotic microscope 21 also includes a computer (not shown) that operates the robotic microscope. The microscopic slide 28 has a label attached to it (not shown) for identification of the slide, such as a commercially available barcode label. The label, which will be referred to herein as a barcode label for convenience, is used to associate a database with the tissue samples on the slide.

Tissue samples, such as the tissue sample 26, can be mounted by any method onto the microscope slide 28. Tissues can be fresh or immersed in fixative to preserve tissue and tissue antigens, and to avoid postmortem deterioration. For example, tissues that have been fresh-frozen, or immersed in fixative and then frozen, can be sectioned on a cryostat or sliding microtome and mounted onto microscope microscope slides. Tissues that have been immersed in fixative can be sectioned on a vibratome and mounted onto microscope slides. Tissues that have been immersed in fixative and embedded in a substance such as paraffin, plastic, epoxy resin, or celloidin can be sectioned with a microtome and mounted onto microscope slides.

A typical microscope slide has a tissue surface area of about 1250 mm². The approximate number of digital images required to cover that area, using a 20× objective, is 12,500, which would require approximately 50 gigabytes of data storage space. In order to make analysis of tissue slides conducive to automation and economically feasible, it becomes necessary to reduce the number of images required to make a determination.

Aspects of the invention are well suited for capturing selected images from tissue samples of multicellular cells in a fixed relationship structures from any living source, particularly animal tissue. These tissue samples may be acquired from a surgical operation, a biopsy, or similar situations where a mass of tissue is acquired. In addition, aspects of the invention are also suited for capturing selected images from tissue samples of smears, cell smears, and bodily fluids.

The robotic microscope 21 includes a high-resolution translation stage (not shown). Microscope slide 28 containing the tissue microarray 24 is automatically loaded onto the stage of the robotic microscope 21. An auxiliary imaging system in the image capture system 20 acquires a single auxiliary digital image of the full microscope slide 28, and maps the auxiliary digital image to locate the individual tissue sample specimens of the tissue microarray 24 on the microscope slide 28.

FIG. 1B illustrates an auxiliary digital image 30 of the tissue microarray 24 that includes an auxiliary level image of each tissue sample in the tissue microarray 24, including an auxiliary tissue sample image 36 of the tissue sample 26 and the barcode. The image 30 is mapped by the robotic microscope 21 to determine the location of the tissue sections within the microscope slide 28. The barcode image is analyzed by commercially available barcode software, and slide identification information is decoded.

System 20 automatically generates a sequence of stage positions that allows collection of a microscopic image of each tissue sample at a first resolution. If necessary, multiple overlapping images of a tissue sample can be collected and stitched together to form a single image covering the entire tissue sample. Each microscopic image of tissue sample is digitized into a first pixel data set representing an image of the tissue sample at a first resolution that can be processed in a computer system. The first pixel data sets for each image are then transferred to a dedicated computer system for analysis. By imaging only those regions of the microscope slide 28 that contain a tissue sample, the system substantially increases throughput. At some point, system 20 will acquire an identification of the tissue type of the tissue sample. The identification may be provided by data associated with the tissue microarray 24, determined by the system 20 using a method that is beyond the scope of this discussion, or by other means.

FIG. 1C illustrates a tissue sample image 46 of the tissue sample 26 acquired by the robotic microscope 21 at a first resolution. For a computer system and method to recognize a tissue constituent based on repeating multi-cellular patterns, the image of the tissue sample should have sufficient magnification or resolution so that features spanning many cells as they occur in the tissue are detectable in the image. A typical robotic pathology microscope 21 produces color digital images at magnifications ranging from 5× to 60×. The images are captured by a digital charge-couple device (CCD) camera and may be stored as 24-bit tagged image file format (TIFF) files. The color and brightness of each pixel may be specified by three integer values in the range of 0 to 255 (8 bits), corresponding to the intensity of the red, green and blue channels respectively (RGB). The tissue sample image 46 may be captured at any magnification and pixel density suitable for use with system 20 and algorithms selected for identifying a structure of interest in the tissue sample 26. Magnification and pixel density may be considered related. For example, a relatively low magnification and a relatively high-pixel density can produce a similar ability to distinguish between closely spaced objects as a relatively high magnification and a relatively low-pixel density. An embodiment of the invention has been tested using 5× magnification and a pixel dimension of a single image of 1024 rows by 1280 columns. This provides a useful first pixel data set at a first resolution for identifying a structure of interest without placing excessive memory and storage demands on computing devices performing structure-identification algorithms. As discussed above, the tissue sample image 46 may be acquired from the tissue sample 26 by collecting multiple overlapping images (tiles) and stitching the tiles together to form the single tissue sample image 46 for processing.

Alternatively, the tissue sample image 46 may be acquired using any method or device. Any process that captures an image with high enough resolution can be used, including methods that utilize other frequencies of electromagnetic radiation other than visible light, or scanning techniques with a highly focused beam, such as an X-ray beam or electron microscopy. For example, in an alternative embodiment, an image of multiple cells within a tissue sample may be captured without removing the tissue from the organism. There are microscopes that can show the cellular structure of human skin without removing the skin tissue. The tissue sample image 46 may be acquired using a portable digital camera to take a digital photograph of a person's skin. Continuing advances in endoscopic techniques may allow endoscopic acquisition of tissue sample images showing the cellular structure of the wall of the gastrointestinal tract, lungs, blood vessels and other internal areas accessible to such endoscopes. Similarly, invasive probes can be inserted into human tissues and used for in vivo tissue sample imaging. The same methods for image analysis can be applied to images collected using these methods. Other in vivo image generation methods can also be used provided they can distinguish features in a multi-cellular image or distinguish a pattern on the surface of a nucleus with adequate resolution. These include image generation methods such as CT scan, MRI, ultrasound, or PET scan.

FIG. 1D illustrates the system 20 providing the tissue image 46 to a computing device 100 in a form of a first pixel data set at a first resolution. The computing device 100 receives the first pixel data set into a memory over a communications link 118. The system 20 may also provide an identification of the tissue type from the database associated with the tissue image 46 using the barcode label.

An application running on the computing device 100 includes a plurality of structure-identification algorithms. At least two of the structure-identification algorithms of the plurality of algorithms are responsive to different tissue types, and each structure-identification algorithm correlating at least one cellular pattern in a given tissue-type with a presence of a structure of interest for the given tissue type. The application selects at least one structure-identification algorithm responsive to the tissue type, and applies the selected algorithm to determine a presence of a structure of interest for the tissue type.

The application running on the computing device 100 and the system 20 communicate over the communications link 118 and cooperatively adjust the robotic microscope 21 to capture a second pixel data set at a second resolution. The second pixel data set represents an image 50 of the structure of interest. The second resolution provides an increased degree to which closely spaced objects in the image can be distinguished from one another over the first resolution. The adjustment may include moving the high-resolution translation stage of the robotic microscope 21 into a position for image capture of the structure of interest. The adjustment may also include selecting a lens 22 having an appropriate magnification, selecting a CCD camera having an appropriate pixel density, or both, for acquiring the second pixel data set at the higher, second resolution.

The application running on the computing device 100 and the system 20 cooperatively capture the second data set. If multiple structures of interest are present in the tissue sample 26, multiple second pixel data sets may be captured from the tissue image 46. The second pixel data set is provided by system 20 to computing device 100 over the communications link 118. The second pixel data set can be have a structure-identification algorithm applied to it for location of a structure of interest, or stored in the computing device 100 along with the tissue type and any information produced by the structure-identification algorithm. Alternatively, the second pixel data set representing the structure of interest 50 may be captured on a tangible visual medium, such as photo sensitive film in a camera or a computer monitor, or printed from the computing device 100 in any type of visual display, such as a monitor or an ink printer, or provided in any other suitable manner. The first pixel data set may then be discarded. The captured image can be further used in a fully automated process of localizing gene expression within normal and diseased tissue, and identifying diseases in various stages of progression. Such further uses of the captured image are beyond the scope of this discussion.

Capturing a high-resolution image of a structure of interest 50 (second pixel data set) and discarding the low-resolution image (first pixel data set) minimizes the amount of storage required for automated processing. Those portions of the tissue sample 26 having a structure of interest are stored. There is no need to save the low-resolution image (first pixel data set) because relevant structures of interest have been captured in the high-resolution image (second pixel data set).

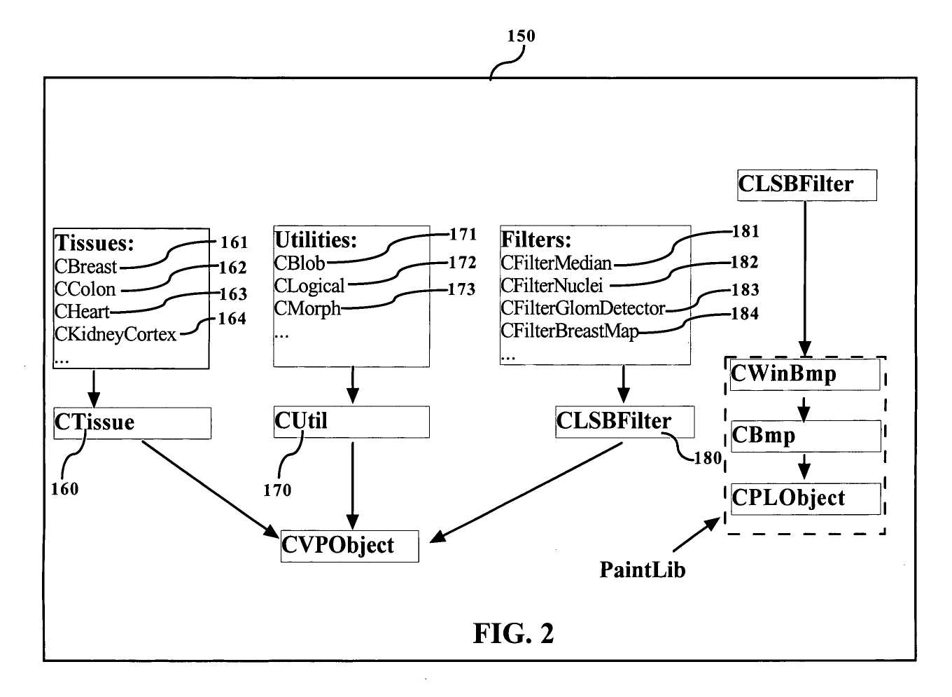

FIG. 2 is a class diagram illustrating several object class families 150 in an image capture application that automatically captures an image of a structure of interest in a tissue sample, according to an embodiment of the invention. The object class families 150 include a tissue class 160, a utility class 170, and a filter class 180. The filter class 180 is also referred to herein as “a plurality of structure-identification algorithms.” While aspects of the application and the method of performing automatic capture of an image of a structure of interest may be discussed in object-orientated terms, the aspects may also be implemented in any manner capable of running on a computing device, such as the computing device 100 of FIG. 1D. In addition to the object class families 150, FIG. 2 also illustrates object classes CVPObject and CLSBImage that are part of an implementation that was built and tested. Alternatively, the structure identification algorithms may be automatically developed by a computer system using-artificial intelligence methods, such as neural networks, as disclosed in U.S. application Ser. No. 10/120,206 entitled Computer Methods for Image Pattern Recognition in Organic Material, filed Apr. 9, 2002.

FIG. 2 illustrates an embodiment of the invention that was built and tested for the tissue types, or tissue subclasses, listed in Table 1. The tissue class 160 includes a plurality of tissue type subclasses, one subclass for each tissue type to be processed by the image capture application. A portion of the tissue type subclasses illustrated in FIG. 2 are breast 161, colon 162, heart 163, and kidney cortex 164.

TABLE 1
Tissue types

| | | Responsive Filter Class |
|---|---|---|
| | | (180) |
| Tissue | | (responsive structure- |
| Type (160) | Tissue Constituents | identification algorithms) |
| Bladder | Surface Epithelium, Smooth | FilterBladderZone |
| | Muscle, Lamina Propria | |
| Breast | Ducts/Lobules, Stroma | FilterBreastMap. |
| | | FilterBreastDucts |
| Colon | Epithelium, Muscularis Mucosa, | FilterColonZone, |
| | Smooth Muscle, Submucosa | |
| Heart | Tissue (generic) | FilterSkeletalMuscle |
| Kidney | Glomerali, PCTS, DCTs, | FilterKidneyCortexMap, |
| Cortex | | FilterGlomDetector, |
| | | FilterTubeDetector |
| Kidney | Ducts | FilterKidneyDetector, |
| Medulla | | FilterDuctDetector |
| Liver | Portal Triad | FilterLiverMap |
| Lung | Alveoli, Respiratory Epithelium | FilterLungMap |
| Lymph Node | Mantle Zone of Lymphoid | FilterLymphnodeMap |
| | Follicle | |
| Nasal | Epithelium | FilterNasalMucosaZone |
| Mucosa | | |
| Placenta | Tissue (generic) | FilterPlacenta |
| Prostate | Glands, Stroma, Epithelium | FilterProstateMap |
| Skeletal | Tissue (generic) | FilterSkeletalMuscle |
| Muscle | | |
| Skin | Epidermis | FilterSkinMap |
| Small | Epithelium, Muscularis Mucosa, | FilterSmIntZone |
| Intestine | Smooth Muscle, Submucosa | |
| Spleen | White Pulp | FilterSpleenMap |
| Stomach | Epithelium, Muscularis Mucosa, | FilterStomachZone |
| | Smooth Muscle, Submucosa | |
| Testis | Leydig Cells | FilterTestisMap |
| Thymus | Lymphocytes, Hassall's | FilterThymusMap |
| | Corpuscles | |
| Thyroid | Follicles | FilterThyroidMap, |
| | | FilterThyroidZone |
| Tonsil | Mantle Zone of Lymphoid | FilterTonsilMap |
| | Follicle, Epithelium | |
| Uterus | Glands, Stroma, Smooth Muscle | FilterUterusZone |

For the tissue types of Table 1, the structure of interest for each tissue type consists of at least one of the tissue constituents listed in the middle column, and may include some or all of the tissue components. An aspect of the invention allows a user to designate which tissue constituents constitute a structure of interest. In addition, for each tissue type of Table 1, the right-hand column lists one or more members (structure-identification algorithms) of the filter class 180 (the plurality of structure-identification algorithms) that are responsive to the given tissue type. For example, a structure of interest for the colon 162 tissue type includes at least one of Epithelium, Muscularis Mucosa, Smooth Muscle, and Submucosa tissue constituents, and the responsive filter class is FilterColonZone. As illustrated by Table 1, the application will call FilterColonZone to correlate at least one cellular pattern formed by the Epithelium, Muscularis Mucosa, Smooth Muscle, and Submucosa tissue constituents to determine a presence of a structure of interest in the colon tissue 162.

A portion of the filter subclasses of the filter class 180 is illustrated in FIG. 2 as FilterMedian 181, FilterNuclei 182, FilterGlomDetector 183, and FilterBreastMap 184. Table 2 provides a more complete discussion of the filter subclasses of the filter class 180 and discusses several characteristics of each filter subclass. The filter class 180 includes both specific tissue-type-filters and general-purpose filters. The “filter intermediate mask format” column describes an intermediate mask prior to operator(s) being applied to generate a binary structure mask.

TABLE 2
Filter Subclasses

| | | | |
|---|---|---|---|
| | | | Filter Intermediate |
| Subclasses | Short Description | Input Format | Mask Format |

Tissue-Specific Filters

| | | | |
|---|---|---|---|
| FilterAdrenalMap | Map regions of the Adrenal | 32 bpp tissue | 32 bpp color map |
| | | image at ≧5x | BLUE: gland |
| | | | tissue |
| | | | GREEN: |
| | | | capsule |
| FilterBladderZone | Map regions of the Bladder | 32 bpp tissue | 32 bpp (R = G = B) |
| | | image at 5x | Coded by gray level |
| FilterBreastDucts | Detect duct | 32 bpp tissue | 8 bpp mask |
| | structures in Breast | image at ≧5x | |
| FilterBreastMap | Map the structures | 32 bpp tissue | 32 bpp color map |
| | of the Breast | image at ≧5x | BLUE: |
| | | | ducts/lobules |
| | | | GREEN: stroma |
| FilterCerebellum | Map the layers of | 32 bpp tissue | 32 bpp color map |
| | the Brain | image at ≧5x | BLUE: lumen |
| | Cerebellum | | GREEN: |
| | | | molecular layer |
| | | | RED: granular layer |
| FilterColonZone | Map the regions of | 32 bpp tissue | 32 bpp (R = G = B) |
| | the Colon | image at 5x | Coded by gray level |
| FilterDuct-Detector | Detect duct | 32 bpp tissue | 32 bpp color map |
| | structures in Kidney | image at ≧5x | BLUE: empty |
| | Medulla | | GREEN: duct + Henle |
| | | | lumen |
| | | | RED: duct |
| | | | lumen |
| FilterGlom-Detector | Detects the | 32 bpp tissue | 32 bpp color map |
| | glomeruli and the | image at ≧5x | BLUE: gloms |
| | Bowman's capsule | | GREEN: |
| | in Kidney Cortex. | | bowman |
| | | | RED: lumen |
| FilterKidney- | Map the structures | 32 bpp tissue | 32 bpp color map |
| CortexMap | of the Kidney | image at ≧5x | BLUE: gloms |
| | Cortex | | MAGENTA: |
| | | | Bowman's |
| | | | capsule |
| | | | GREEN: DCT |
| | | | RED: PCT |
| FilterKidney- | Map the structures | 32 bpp tissue | 32 bpp color map |
| MedullaMap | of the Kidney | image at ≧5x | GREEN: duct + Henle |
| | Medulla | | lumen |
| | | | RED: duct |
| | | | lumen |
| FilterLiverMap | Map the locations | 32 bpp tissue | 32 bpp color map |
| | of the portal triads | image at ≧5x | BLUE: portal |
| | in Liver | | triad |
| | | | GREEN: portal |
| | | | triad |
| | | | RED: portal triad |
| FilterLungMap | Map the alveoli and | 32 bpp tissue | 32 bpp color map |
| | respiratory | image at ≧5x | BLUE: alveoli |
| | epithelium in Lung | | GREEN: |
| | | | epithelium |
| FilterLymphnode- | Map the structures | 32 bpp tissue | 32 bpp color map |
| Map | of the Lymph Node | image at ≧5x | BLUE: mantle |
| | | | zone of |
| | | | lymphoid follicle |
| FilterNasal- | Map the regions of | 32 bpp tissue | 32 bpp (R = G = B) |
| MucosaZone | the nasal mucosa | image at 5x | Coded by gray |
| | | | level |
| FilterPlacenta | Map tissue in | 32 bpp tissue | 8 bpp mask |
| | Placenta | image | |
| FilterProstateMap | Detects the glands, | 32 bpp tissue | 32 bpp color map |
| | stroma and | image at ≧5x | BLUE: glands |
| | epithelium in | | GREEN: stroma |
| | Prostate | | RED: epithelium |
| FilterSkeletal- | Maps the tissue | 32 bpp tissue | 8 bpp mask |
| Muscle | areas in skeletal | image at ≧5x | |
| | muscle | | |
| FilterSkinMap | Map the structures | 32 bpp tissue | 32 bpp color map |
| | of the Skin | image at ≧5x | BLUE: epidermis |
| | | | GREEN: |
| | | | epidermis |
| | | | RED: epidermis |
| FilterSmIntZone | Map the regions of | 32 bpp tissue | 32 bpp (R = G = B) |
| | the Small Intestine | image at 5x | Coded by gray |
| | | | level |
| FilterSpleenMap | Map the structures | 32 bpp tissue | 32 bpp color map |
| | of the Spleen | image at ≧5x | BLUE: white |
| | | | pulp |
| FilterStomachZone | Map the regions of | 32 bpp tissue | 32 bpp (R = G = B) |
| | the Stomach | image at 5x | Coded by gray |
| | | | level |
| FilterTestisMap | Map the structures | 32 bpp tissue | 32 bpp color map |
| | of the Testis | image at ≧5x | BLUE: interstitial |
| | system | | region |
| | | | GREEN: Leydig |
| | | | cells |
| | | | RED: |
| | | | seminiferous |
| | | | tubules |
| FilterThymusMap | Map the | 32 bpp tissue | 32 bpp color map |
| | lymphocyte areas | image at ≧5x | BLUE: |
| | and Hassall's | | lymphocytes |
| | corpuscles in | | GREEN: |
| | Thymus | | Hassall's |
| FilterThyroidMap | Map the Follicles in | 32 bpp tissue | 8 bpp mask |
| | Thyroid | image at ≧5x | |
| FilterThyroidZone | Map the Follicles in | 32 bpp tissue | 32 bpp (R = G = B) |
| | Thyroid | image at 5x | Coded by gray |
| | | | level |
| FilterTonsilMap | Map the structures | 32 bpp tissue | 32 bpp color map |
| | of the Tonsil | image at ≧5x | BLUE: mantle |
| | | | zone of |
| | | | lymphoid follicle |
| FilterTube-Detector | Detects the tubule | 32 bpp tissue | 32 bpp color map |
| | structures in the | image at ≧5x | BLUE: empty |
| | Kidney Cortex and | | GREEN: PCT + DCT |
| | classifies them as | | lumen |
| | PCT or DCT | | RED: DCT |
| | | | lumen |
| FilterUterusZone | Map the regions of | 32 bpp tissue | 32 bpp (R = G = B) |
| | the Uterus | image at 5x | Coded by gray |
| | | | level |

General-Purpose Filters

| | | | |
|---|---|---|---|
| FilterDistanceMap | Distance transform | 8 or 32 bpp | 8 or 32 bpp gray |
| | | binary image | level distance |
| | | | map |
| FilterDownSample | Down-samples an | 8 or 32 bpp | 8 or 32 bpp |
| | image by binning | image | down-sampled |
| | | | image |
| FilterDSIntensity | Computes intensity | 8 or 32 bpp | 8 bpp image |
| | image by averaging | image | (may be down- |
| | the R, G & B with | | sampled) |
| | optional | | |
| | simultaneous down | | |
| | sampling | | |
| FilterEnhance | Fast digital | 8 or 32 bpp | 8 or 32 bpp |
| | enhancement (RGB | image | enhanced |
| | to RGB) | | image |
| FilterEpithelium | Detects epithelial | 32 bpp tissue | 8 bpp epithelium |
| | cells in tissues. | image | mask |
| | Parameters are set | | |
| | through access | | |
| | functions. | | |
| FilterErodeNuclei | Thresholded | 8 or 32 bpp | 8 or 32 bpp |
| | erosion of | binary image | eroded binary |
| | components in | | image |
| | binary image | | |
| FilterExpand- | Thresholded | 8 or 32 bpp | 8 or 32 bpp |
| Nuclei | expansion of | binary image | expansion |
| | components binary | | binary image |
| | image | | |
| FilterFastAverage | Fast averaging filter | 8 or 32 bpp | 8 or 32 bpp local |
| | with optional | image | average image |
| | normalization | | |
| FilterFractal- | Computes the | 8 or 32 bpp | 8 or 32 bpp |
| Density | fractal density map | binary image | fractal density |
| | of a black-and-white | | image |
| | image | | |
| FilterIPLRotate | Rotates image | 32 bpp image | 32 bpp rotated |
| | | | image |
| FilterJoin- | Morphologically | 8 or 32 bpp | 8 or 32 bpp |
| Components | joins components | binary image | binary image |
| | that are close | | |
| | together | | |
| FilterMask | Extracts a 8 bpp | 32 bpp image | 8 bpp image |
| | image from a 32 bpp | | |
| | bitmap | | |
| FilterMedian | Computes median | 8 or 32 bpp | 8 or 32 bpp |
| | filter | image | filtered image |
| FilterNuclei | Computes a nuclei | 32 bpp tissue | 8 bpp nuclei |
| | mask using a | image at ≧5x | mask |
| | segmentation | | |
| | technique. | | |
| FilterResizeMask | Resizes binary | 8 or 32 bpp | 8 or 32 bpp |
| | images | binary image | binary image |
| FilterROISelector | Finds candidate | 8 bpp binary | Places the ROI |
| | regions of interest | image | information in |
| | based a supplied | | the ROIlist data |
| | structure mask. | | structure. |
| FilterSegment | Computes a | 32 bpp tissue | 32 bpp color map |
| | segmented image | image | BLUE: dark |
| | where nuclei, white | | pixels |
| | space and Vector | | GREEN: white |
| | red are identified. | | pixels |
| | | | RED: Vector red |
| FilterSuppressVR | Suppresses or | 32 bpp tissue | 32 bpp tissue |
| | removed Vector | image | image |
| | Red content from | | |
| | the tissue image | | |
| FilterTextureMap | Computes the | 8 or 32 bpp | 8 bpp texture |
| | variance-based | image | map |
| | texture map | | |
| FilterTissueMask | Computes a mask | 32 bpp tissue | 8 bpp tissue |
| | that indicates the | image | mask |
| | location of the | | |
| | tissue in an image | | |
| | (i.e. not white | | |
| | space) | | |
| FilterWhiteSpace | Computes a white | 32 bpp tissue | 8 bpp white- |
| | space mask using a | image | space mask |
| | user-selectable | | |
| | method. | | |
| FilterZoom | Fast digital zoom | 8 or 32 bpp | 8 or 32 bpp |
| | (RGB to RGB) | image | digitally zoomed |
| | | | image |

For example, when determining the presence of a structure of interest for the colon 162 tissue type, the application will call the responsive filter class FilterColonZone. Table 2 establishes that the FilterColonZone will map the regions of the Colon with 32 bpp using a first pixel data set representing the tissue sample at a resolution image magnification of 5×, and will compute an intermediate mask at 32 bpp (R=G=B) coded by gray level. An aspect of the invention is that the subfilters of the filter class 180 utilizes features that are intrinsic to each tissue type, and do not require the use of special stains or specific antibody markers.

Tables 3 and 4 describe additional characteristics of the filter subclasses of the filter class 180.

### Table 3 Tissue-Specific Filters/Structure-Identification Algorithms

FilterAdrenalMap

This program recognizes glandular tissue (cortex and medulla), and capsule tissue, using nuclei density as the basic criterion. In the case of the cortex, the nuclei density is computed from a nuclei mask that has been filtered to remove artifacts, and the result is morphologically processed. For capsule detection, the glandular tissue area is removed from the total tissue image, and the remaining areas of tissue are tested for the correct nuclei density, followed by morphological processing. The resulting map is coded with blue for glandular tissue and green for capsule tissue.

FilterBladderZone

The program recognizes three zones: surface epithelium, smooth muscle and lamina propria. The algorithm first segments the input image into three classes of regions: nuclei, cytoplasm, and white space. Based on the segmentation result, the “density map” for each class is calculated and used to find the potential locations of the target zones. Zones are labeled with the following gray levels: Surface Epithelium—50, Smooth Muscle—100, and Lamina Propria—150

FilterBreastDucts

The input is a breast image and the output is a binary mask indicating the ducts. The routine finds epithelium in the breast by successively filtering the nuclei. The key observation is that epithelia are very hard to separate and rather large. Building on that observation, nuclei are discarded first by size, i.e., the smallest nuclei are eliminated. Larger nuclei are discarded if they are too elongated. Isolated nuclei are also discarded. The remaining nuclei are then joined using center of mass option of FilterJoinComponents. A second pass eliminates components that are thin. The remaining components are classified as ducts.

FilterBreastMap

The input is a breast image and the output is a color map, with blue denoting ducts, green stroma and black adipose or lumen. The ducts are found using FilterBreastDucts. The remaining area can be stroma, lumen (white space) or adipose. The adipose has a beautiful ‘lattice-like’ structure; its complement is many small lumen areas. Hence growing such areas will encase the adipose. The results of this region growing together with the white space (given by the FilterSegment program) yield the complement of the stroma and ducts. Hence the stroma is determined.

FilterColonZone

This program segments the input image into three classes of regions: nuclei, cytoplasm, and white space. Based on the segmentation result, the “density map” for each class is calculated. Using the density maps produced above, find the potential locations of the “target zones”: epithelium, smooth muscle, submucosa, and muscularis mucosa. Each potential target zone is then analyzed with some tools for local statistics, and morphological operations in order to get a more precise estimation of its location and boundary. Regions are labeled with the following gray levels: Epithelium—50, Smooth muscle—100, Submucosa—150, and Muscularis Mucosa—200.

FilterDuctDetector

This filter is designed to detect and identify candidate collecting ducts in the kidney medulla. It is completed in three major parts: image layer segmentation, shape filters applied to measure candidate object properties, and finally an analysis test to identify the ducts. The segmentation involves white-space detection with removal of small areas and then nuclei detection. Distance filters are applied to compute the distance between candidate lumen and the closest surrounding nuclei. The final analysis identifies ducts that match a specific criteria for distance between nuclei and lumen and nuclei to lumen ratio.

FilterGlom Detector

This filter is designed to detect and identify candidate glomeruli and their corresponding Bowman's capsules. It is completed in three major parts: image layer segmentation, shape filters applied to measure candidate object properties, and finally an analysis test to identify the glomeruli. The segmentation involves white-space detection with removal of small areas, nuclei and Vector red detection (if present in the image). Shape filters such as compactness and form factor are applied next to measure these properties for each lumen. A radial ring is positioned around each lumen then nuclei density and Vector red density scores are computed. The final analysis uses criteria for compactness, form factor, nuclei density and Vector red density to identify candidate glomeruli regions.

FilterKidneyCortexMap

This filter is designed to map the glomeruli, distal and proximal convoluted tubules of the kidney cortex. It calls FilterTubeDetector and FilterGlomDetector, and combines the results to create one structure mapped RGB image with glomeruli in blue, Bowman's capsule as magenta, distal convoluted tubules as green and proximal convoluted tubules as red.

FilterKidneyMedullaMap

This filter is designed to map the collecting ducts of the kidney medulla. It calls FilterDuctDetector to create one structure mapped RGB image with ducts and Henle lumen as green and duct lumen as red.

FilterLiverMap

Identifies the location of the portal triad by the presence of ducts and lack of nuclei. The portal triad structures are coded into all channels.

FilterLungMap

Maps the tissue areas corresponding to the alveoli and respiratory epithelium. Alveoli detection is done by morphological filtering of the tissue mask. Respiratory epithelium is detected by applying a double threshold to the nuclei density and filtering out blobs of the wrong shape. The result is coded with blue for alveoli and green for epithelium.

FilterLymphnodeMap

Maps the tissue areas corresponding to lightly stained spherical lymphoid follicles surrounded by darkly stained mantle zones. The mantle zone of a follicle morphologically corresponds to areas of high nuclei density. Thresholding the nuclei density map is the primary filter applied to approximate the zones. To improve detection of the mantle zone, the areas corresponding to low nuclei density areas (e.g., germinal center and surrounding cortex tissue) are suppressed in the original image. A second segmentation and threshold are applied to the suppressed image to produce the final zones. The map is coded blue for mantle zone.

FilterNasalMucosaZone

There three types of substructures in nasal mucosa tissues are of interest: respiratory epithelium, sub-mucosa glands, and inflammatory cells. The latter can not be detected at 5× magnification. To detect epithelium and glands, the image is first segmented into three classes of regions: nuclei, cytoplasm, and white space, and the “density map” is computed for each, followed by morphological operations. Regions are labeled with the following gray levels: Epithelium—50; Glands—100

FilterPlacenta

This function maps the location of all tissue by computing the complement of the texture-based white-space mask on the Vector red-suppressed image (see FilterSuppressVR). The output is an 8 bpp mask image.

FilterProstateMap

The input is a prostate image and the output is a color map indicating the glands as blue, stroma as green and epithelium as red. The glands are bounded by epithelium. The epithelium is found much the same as in breast. Isolated, elongated and smaller nuclei are eliminated. The compliment of the image remaining epithelia consists of several components. A component is deemed to be a gland if the nuclear density is sufficiently low; otherwise it is classified a stroma/smooth muscle component and intersected with the tissue mask from FilterTissueMask to give the stroma.

FilterSkeletalMuscle

This function maps the location of all tissue by computing the complement of the texture-based white-space mask on the Vector red-suppressed image (see FilterSuppressVR) after down-sampling to an equivalent magnification of 1.25×. The output is an 8 bpp mask image.

FilterSkinMap

This program recognizes the epidermis layer by selecting tissue regions with nuclei that have a low variance texture in order to avoid “crispy” connective tissue areas. A variance-based segmentation is followed by morphological processing. Regions with too few nuclei are then discarded, giving the epidermis. The resulting mask is written into all channels.

FilterSmIntZone

This program segments the input image into three classes of regions: nuclei, cytoplasm, and white space. Based on the segmentation result, the “density map” for each class is calculated. Using the density maps produced above, find the potential locations of the “target zones”: epithelium, smooth muscle, submucosa, and muscularis mucosa. Each potential target zone is then analyzed with some tools for local statistics, and morphological operations in order to get a more precise estimation of its location and boundary. Regions are labeled with the following gray levels: Epithelium—50, Smooth muscle—100, Submucosa—150, and Muscularis Mucosa—200.

FilterSpleenMap

Maps the tissue areas corresponding to white pulp, which contains lymphoid follicles. The mantle zone of a splenic follicle morphologically corresponds to areas of high nuclei density common in lymphoid tissues. Thresholding the nuclei density map is the primary filter applied to approximate the zones. To improve detection of the mantle zone, the areas corresponding to low nuclei density areas (e.g., germinal center and surrounding red pulp and other parenchyma tissue) are suppressed in the original image. A second segmentation and threshold are applied to the suppressed image to produce the final zones. The map is coded with blue for white pulp.

FilterStomachZone

Segments the input image into three classes of regions: nuclei, cytoplasm, and white space. Based on the segmentation result, the “density map” for each class is calculated. Using the density maps produced above, find the potential locations of the “target zones”: epithelium, smooth muscle, submucosa, and muscularis mucosa. Each potential target zone is then analyzed with some tools for local statistics, and morphological operations in order to get a more precise estimation of its location and boundary. Regions are labeled with the following gray levels: Epithelium—50, Smooth muscle—100, Submucosa—150, and Muscularis Mucosa—200.

FilterTestisMap

This filter is designed to map the interstitial region and Leydig cells of the testis. The initial step is to segment the image into nuclei and white-space/tissue layer images. Next the nuclei density is computed from the nuclei image and then thresholded. The initial interstitial region is found by taking the “exclusive OR” (or the absolute difference) of the tissue/white-space image and the nuclei density image.

The candidate Leydig cell regions are found by taking the product of the original image and the interstitial region. The candidate Leydig cells are found by taking the product of the previous Leydig cell region image and the nuclei density image. The final cells are identified by thresholding using a size criteria. The resulting structure map shows the interstitial regions as blue and the Leydig cells as green.

FilterThymusMap

Maps the tissue areas corresponding to cortex and Hassall's corpuscles. The cortex regions are those of high nuclei density, and can be used to find lymphocytes. Because positive identification of Hassall's corpuscles at 5× magnification is currently not possible, the program produces a map of potential corpuscles for the purpose of ROI selection. Potential corpuscles are regions of low nuclei density that is not white-space and is surrounded by medulla (a region of medium nuclei density). Size and shape filtering is done to reduce false alarms. The result is coded with blue for lymphocytes and green for Hassall's corpuscles.

FilterThyroidMap

Maps the follicles in the Thyroid by selecting nuclei structures that surround areas which are devoid of nuclei and are within the proper size and shape range. An 8 bpp follicle mask is produced.

FilterTonsilMap

Maps the tissue areas corresponding to lightly stained spherical lymphoid follicles surrounded by darkly stained mantle zones. The mantle zone of a follicle morphologically corresponds to areas of high nuclei density common in lymphoid tissues. Thresholding the nuclei density map is the primary filter applied to approximate the zones. Vector red suppression is applied as a pre-processing step to improve nuclei segmentation. To improve detection of the mantle zone, the areas corresponding to low nuclei density areas (e.g., germinal center and surrounding cortex tissue) are suppressed in the original image. A second segmentation and threshold are applied to the suppressed image to produce the final zone. The map is coded with blue for mantle zone.

FilterTubeDetector

This filter is designed to detect and identify candidate distal convoluted tubules and proximal convoluted tubules in the kidney cortex. It is completed in three major parts: image layer segmentation, shape filters applied to measure candidate object properties, and finally a analysis test to identify the ducts. The segmentation involves white-space detection with removal of small areas and nuclei detection. Distance filters are applied to compute the distance between candidate lumen and the closest surrounding nuclei. The final analysis identifies distal convoluted tubules that match a specific criteria for distance between nuclei and lumen and nuclei to lumen ratio. The rejected candidate tubules are identified as Proximal Convoluted tubules.

FilterUterusZone

This program segments the input image into three classes of regions: nuclei, cytoplasm, and white space. Based on the segmentation result, the “density map” for each class is calculated. Using the density maps produced above, find the potential locations of the “target zones”: stroma, glands, and muscle. Each potential target zone is then analyzed with some tools for local statistics, and morphological operations in order to get a more precise estimation of its location and boundary. Regions are labeled with the following gray levels: Stroma—50, Glands—100, and Muscle—150.

End of Table 3.

### Table 4 General-Purpose Filters/Algorithms

FilterDistanceMap

Function to compute distance transform using morphological erosion operations. Works with 8 or 32 bpp images. In the 32 bpp case, the BLUE channel is used. The output is scaled to the [0,255] range. The true maximum distance value is saved in the CLSBImage object.

FilterDownSample

This function down-samples the source image by a constant factor by averaging over pixel blocks. The alignment convention is upper-left. A sampling factor of 1 results in no down-sampling. If the source bitmap has dimensions that are not an integer multiple of the sampling factor, the remaining columns or rows are averaged to make the last column or row in the destination bitmap. The source bitmap can be 8 or 32 bits per pixel. The result is placed in a new bitmap with the same pixel depth and alpha channel setting as the source bitmap. The sampling factor can be set in the constructor or by the function SetSampling. Default is 2.

FilterDSIntensity

This function computes the gray-scale image by average of red, green and blue. The result is placed in a new bitmap with 8 bits per pixel and the same alpha setting as the source. Also provides simultaneous down-sampling by a constant factor (see FitlerDownSample). The sampling factor can be set in the constructor or by the function SetSampling. Default is 1 (no down-sampling).

FilterEnhance

This function enhances the image that was magnified by FilterZoom. It uses the IPL (Intel's Image Processing Library) to smooth the edges.

FilterEpithelium

This function applies a generic algorithm to segment the epithelial regions. Various parameters are empirically determined for each tissue. Output is an 8 bpp mask that marks the epithelium.

FilterErodeNuclei

A square structural element of given size is used to erode the nuclei mask subject to a threshold. If the number of ON pixels is less than the threshold, all the pixels inside the element are turned OFF. Otherwise they are left as they were. The structural element size and threshold value can be passed to the constructor or set through access functions. Works with 8 or 32 bpp bitmaps. For 32 bpp, the blue channel is used.

FilterExpandNuclei

A square structural element of given size is used to dilate the nuclei mask subject to a threshold. If the number of ON pixels is greater than the threshold, all the pixels inside the element are turned ON. Otherwise they are left as they were. The structural element-size and threshold value can be passed to the constructor or set through access functions. Works with 8 or 32 bpp bitmaps. For 32 bpp, the blue channel is used.

FilterFastAverage

Function to filter image using a square averaging mask of size 2*S+1. If normalization is set to ON, the input image is treated as binary and the output is scaled to be in the range [0,255], where the value 255 corresponds to a pixel density of 1.0 over the entire window. Window size and normalization can be set at the constructor or by access functions. Works with 8 or 32 bpp bitmaps. In the 32 bpp case, a grayscale image is obtained by taking the mean square average of the three color channels.

FilterFractalDensity

Fractal descriptors measure the complexity of self-similar structures across different scales. The fractal density (FD) mapping measures the local non-uniformity of nuclei distribution and is often termed the fractal dimension. One method for implementing the FD is the box-counting approach. We implement one variation of this approach by partitioning the image into square boxes of size L×L and counting the number of N(L) of boxes containing at least a portion of the shape. The FD can be calculated as the absolute value of the slope of the line interpolated to a log(N(L))×log(L) plot.

The sequence of box sizes, starting from a given size L over a given pattern in the image, is usually reduced by ½ from one level to the next. FD measurements 2>FD>1 typically correspond to the most fractal regions, implying more complex shape information.

FilterIPLRotate

This function rotates the image with Cubic interpolation (default). It uses the IPL (Intel's Image Processing Library) RotateCenter call.

FilterJoinComponents

This filter contains 2 methods for joining components; typically this is used to join nuclei. The inputs are a binary image, a size, number of passes and an output.

Line Method: A square window, W, with edge 2*S+1 is placed at each point in the image. If the center pixel of W is not zero then it is joined to each non-zero pixel in W. That is, each pixel along the straight line joining the center pixel is set to 1.

Centroid Method: A square window, W, with edge 2*S+1 is placed at each point in the image. If the center pixel of W is equal to zero then the center of mass of the non-zero pixels is calculated and set to 1.

FilterMask

Function to extract a binary image (mask) from a bitmap by applying a threshold to a given channel of the source bitmap. The source bitmap can be 8 or 32 bits per pixel. The destination bitmap is 8 bits per pixel (a single plane). Multiple constructors exist to apply thresholds in different ways (see below). The destination mask can be optionally inverted.

FilterMedian

Function to apply a median-filter of specified size. Calls IPL's median filter function. Kernel size in the form (width, height) and can be passed to the constructor or set through an access function. Default size is 5×5. A 5×5 kernel results in a square window of size 25, and center (3,3). Works with 8 or 32 bpp bitmaps.

FilterNuclei

Function to segment nuclei from tissue image at an arbitrary magnification. The program calls FilterSegment to quantize the input image into three gray levels, and applies a color test to the lowest (darkest) level to obtain the nuclei mask. Output is an 8 bpp bitmap. The constructor takes 3 (optional) parameters that are passed to FilterSegment to initialize the algorithm (see FitlerSegment for discussions).

FilterResizeMask

Function to change the size of a binary mask. Size can be increased or decreased arbitrarily. When down-sampling, the bitmap is sampled at the appropriate row and column sampling factors. When up-sampling, the new bitmap is created by expanding each pixel into a block of appropriate size and then applying a median filter of the same size to smooth any artifacts. The dimensions of the new bitmap can be provided to the constructor or set by the SetNewSize function.

FilterROISelector

Function to select regions of interest (ROIs) from a binary mask bitmap. An averaging filter is used to create a figure of merit (FOM) image from the binary mask. The ROI selector divides the image into a grid such that the number of grid elements is slightly larger than the number of desired ROIs. Each grid element is then subdivided and the element with the highest average FOM is subsequently subdivided until the pixel level is reached, resulting in an ROI center. If ROI dimensions are greater than zero, the centers are then shifted so that no ROI pixels fall outside the image. The ROIs are then scored by calculating the fraction of the ROI pixels that overlap the source binary mask, and then sorted by decreasing score. If either of the ROI dimensions is zero, the FOM values are used as the score. Finally, overlapping ROIs are removed, keeping the higher-scoring ones. The ROI information is placed in the CLSBImage object's ROI list

FilterSegment

Function to segment tissue image into three gray levels using a modified k-means clustering method. Initialization is controlled by three parameters passed to the constructor or set using access functions:

NucEst—Fraction of dark pixels used to compute initial dark mean.

WhtEst—Fraction of bright pixels used to compute initial bright mean.

Center—Parameter to skew the location of the center mean. A value of 0.5 places it in the middle point between the dark and bright means.

Two other parameters are used to control the treatment of Vector red pixels:

VrEst1—Gray level difference between red and blue for Vector red test.

VrEst2—Size of expansion structural element for initial Vector red mask.

By default, statistics are computed using all the image pixels (GLOBAL). The program also has the option of using LOCAL statistics by dividing the image into overlapping blocks and performing the segmentation on a block-by-block basis. The functions Local( ) and Global( ) are used to set the behavior.

The result is returned as a color map with the dark pixels in blue, white-space pixels in green and Vector red pixels in red.

FilterSuppressVR

Function to suppress the Vector red content in a tissue image. An optional parameter in the range [0,1] sets the resulting VR level relative to the original, with 0 corresponding to complete suppression and 1 to no suppression. Note: a value of 1 will in general not produce the original image exactly. The output is a new RGB image.

FilterTextureMap

Function to compute variance-based texture image (map) from gray-scale source image. If input bitmap is 32 bits per pixel (RGB), then the intensity image is obtained using FilterDSIntensity. The texture map is computed using the Intel IPL library. An optional integer input argument is provided Defines the size of a square local region in the image (i.e. the scale of interest). Given as the length of the side of the square in pixels. Default is 32.

FilterTissueMask

Function to compute mask that marks the location where tissue is present in the image. A texture map at 5× magnification is used to obtain an initial mask. Using the mean and standard deviation of the pixel intensities from the initial masked image, an intensity threshold is computed by the formula: t=mean−gain*(standard deviation). The intensity image is then thresholded, producing a second mask. The final tissue mask is obtained by combining the re-sampled texture mask (to the original magnification) and the intensity mask so that a pixel is marked as “tissue” if both the texture is high and the intensity is low. Otherwise it is white-space. The gain for the intensity threshold can be set in the constructor (default is 2.0) or through an access function.

FilterWhiteSpace

Function to mask white-space in a tissue image. Two methods are provided: Texture and 3Color. The Texture method calls FilterTissueMask and inverts the result to provide a white-space mask. The 3Color method calls FilterSegment and extracts the image plane associated with white-space. In both cases the output is an 8 bit per pixel bitmap. The method is selected by calling the member functions SetMethodTexture( ) or SetMethod3Color( ) with the appropriate parameters prior to calling Apply (or ApplyInPlace). For a discussion of the parameters, see the corresponding Filter. The default method is Texture.

FilterZoom

This function zoom the image with Cubic interpolation (default). It uses the IPL (Intel's Image Processing Library) Zoom.

End of Table 4.

Continuing the example of determining the presence of a structure of interest for the colon 162 tissue type, as discussed in Table 4 the filter subclass FilterColonZone will segment the input image into three classes of regions: nuclei, cytoplasm, and white space. Based on the segmentation results, the “density map” for each class is calculated. Using the density maps produced above, find the potential locations of the “target zones”: epithelium, smooth muscle, submucosa, and muscularis mucosa. Each potential target zone is then analyzed with some tools for local statistics, and morphological operations in order to get a more precise estimation of its location and boundary. Regions are labeled with the following gray levels: Epithelium—50, Smooth muscle——100, Submucosa—150, and Muscularis Mucosa—200.

Table 5 discusses yet further characteristics of structure-identification algorithms responsive to each tissue type of the tissue class 160:

### Table 5

Epithelium

Implementation: FilterEpithelium

Several tissues contain epithelial cells, which can be recognized by the spatial arrangement of the nuclei. Epithelial cells are often located close to each other. Together with their associated cytoplasm, the epithelial nuclei form rather “large” regions. The generic epithelium algorithm first segments the image to obtain a nuclei map and a cytoplasm map. Each “nuclei+cytoplasm” region is assigned a distinct label using a connected component labeling algorithm. Based on such labeling, it is possible to remove “nuclei+cytoplasm” regions that are too small, with the “potential epithelial” regions thus outlined. The region size threshold is empirically determined for different tissues. After obtaining the “potential epithelial” regions, an object shape operator is applied to remove those regions that have very “spiked” boundaries.

Bladder

Implementation: FitlerBladderZone

The Bladder mapping algorithm recognizes three zones: surface epithelium, smooth muscle and lamina propria. The algorithm first segments the input image into three classes of regions: nuclei, cytoplasm, and white space. Based on the segmentation result, the “density map” for each class is calculated and used to find the potential locations of the target zones.

Surface Epithelium

To find the surface epithelium regions, the steps are as follows:
1. The nuclei density map is thresholded using the Otsu method. The areas where nuclei density exceeds the threshold value are labeled as potential epithelium regions. The background (non-tissue) regions are also labeled by thresholding the nuclei density map and retaining the areas where the nuclei density is under the threshold.
2. A size-based filter is applied to clean up the spurious background areas.
3. A morphological dilate operation is applied to the background blobs to “fatten” them so that they overlap with the potential epithelium. The potential epithelium that intersect, or are otherwise connected with, the background can now be labeled as surface epithelium.
4. A size-based filter is applied to clean up the surface epithelium areas.

Smooth Muscle

To find the muscle regions, the steps are:
1. Label all the tissue areas that are not epithelial regions as the potential muscle regions.
2. Perform size-based filtering to remove the spurious muscle regions.
3. Apply a morphological dilation operator to make the muscle regions “fatter”. This is to create some overlap between the muscle regions and its neighboring blobs.
4. If a blob representing a muscle region is next to an epithelium, this blob is removed as no muscle is to be adjacent to surface epitheliums. The result is an estimate of muscle regions.

Lamina Propria

The lamina propria regions are always between the surface epithelium and smooth muscle. They are located by labeling all tissue areas that are neither epithelium or muscle as the potential lamina propria regions, and applying size-based filtering to remove the spurious lamina propria. What remains is the estimate for lamina propria.

Breast

Implementation: FilterBreastMap

Three structures are recognized in Breast: ducts, lobules and stroma. Because of their proximity and the difficulty in discriminating between ducts and lobules, they are lumped together in a single recognition category for the purpose of ROI selection.

Ducts/Lobules

Implementation: FilterBreastDucts

Ducts and Lobules are small structures that consist of a white-space region surrounded by a ring of epithelial cells. All epithelium in Breast surrounds ducts or lobules, and so they can be found by simply locating the epithelial cells. The overall strategy is to compute the nuclei mask and then separate the epithelial from the non-epithelial nuclei.

The key observation is that epithelia are very hard to separate and rather large. Building on that observation, nuclei are discarded first by size, i.e., the smallest nuclei are eliminated. Larger nuclei are discarded if they are too elongated. Isolated nuclei are also discarded. The remaining nuclei are then joined using the center of mass method. A second pass eliminates components that are thin. The remaining components are classified as ducts.

Discarding Isolated Nuclei:

For tissues such as breast or prostate the principal difference between epithelial and non-epithelial nuclei is that the latter are isolated, so that the boundary of a “typical” neighborhood (window) around a non-epithelial nucleus will not meet any other nuclei. Such a condition translates directly into an algorithm. Given a binary nuclei mask, a window is placed about each nucleus. The values of the boundary of the window are then summed. If that sum is 0 (no pixels are turned “on”), the nucleus is classified as non-epithelial and epithelial if the sum is not zero.

Stroma

Once the ducts are found, the remaining area can be stroma, lumen (white space) or adipose. The adipose has a “lattice-like” structure; its complement is many small white-space areas. Hence, growing such areas will encase the adipose. The results of this region growing together with the white space (given by the Segment program AutoColorV2) yield the complement of the stroma and ducts. Hence the stroma is determined.

Colon, Small Intestine and Stomach

Implementation: FilterColonZone, FilterSmIntZone, FilterStomachZone

Structure recognition algorithms for Colon, Small Intestine and Stomach share common key processing steps. They only differ in parameter selection and some minor processes. In all cases, the image is first segmented into three classes of regions: nuclei, cytoplasm, and white space. Based on the segmentation result, the “density map” for each class is calculated. Using the density maps produced above, find the potential locations of the “target zones”: epithelium, smooth muscle, submucosa, and muscularis mucosa.

Epithelium

To obtain the epithelium regions, the Otsu threshold technique is applied to the nuclei density map. The regions where the nuclei density exceeds the Otsu threshold value are classified as potential epithelium. Among the potential epithelium regions, we apply an “isolated blob removal” process, which removes the isolated blobs for a given range of sizes, and within certain ranges of “empty” neighborhood. The next step is to invoke a shape filter that removes the blobs that are too “elongated” based on the eigen-axes of their shapes. A morphological dilation then smoothes the edges of the remaining blobs. The result of this sequence of operations is the epithelium regions.

Submucosa

To find the submucosa regions, a variance map of the gray-scale copy of the original image is first produced. The Otsu threshold is then applied to the variance map. It segments out the potential submucosa and epithelium regions by only retaining the portion of the variance map where the variance exceeds the Otsu threshold values. Since the submucosa regions are disjoint with epithelium, the latter can be removed, and a potential submucosa map is thus produced. A size-based filter is then applied to remove blobs under or exceed certain ranges. The final sub-mucosa regions are thus obtained.

Smooth Muscle

To find the potential muscle regions, the Otsu threshold is applied to the cytoplasm density map. The regions of the map where the density values exceed the threshold value are labeled as the initial estimate for potential muscle regions. After excluding the epithelium and submucosa regions from the potential muscle regions, an isolated blob remover is used to filter out the blobs that are too large or too small and with sufficiently “empty” neighbor regions. This sequence of operations results in the final muscle map.

Muscularis Mucosa

The muscularis mucosa regions are always adjacent to the epithelium and submucosa regions. The first step is to find the boundaries of the epithelium and perform a region growing operation from these epithelium boundaries. The intersections between the regions grown from epithelium and the submucosa are labeled as the muscularis mucosa.

Heart and Skeletal Muscle

Implementation: FilterSkeletalMuscle

No specific structures need be found in Heart and Skeletal Muscle. A generic tissue finder algorithm is used to avoid large white-space areas. The algorithm consists of the following steps:
1. The image is down-sampled to an equivalent magnification of 1.25× for both speed and to capture the large-scale texture information.
2. The Vector red signature is suppressed to avoid false alarms in cases on non-specific binding to the glass.
3. The white-space mask is computed using the Texture-Brightness method and the mask is inverted (positive becomes negative and negative becomes positive).
4. A median filter is applied to “smooth out” noisy areas such as tiny white-space holes in tissue and small specks of material in the white-space. This has the effect of improving the quality of the ROI selection results.
5. The resulting mask is re-sampled to match the size of the original image.
   Kidney Cortex

Implementation: FilterKidneyCortexMap

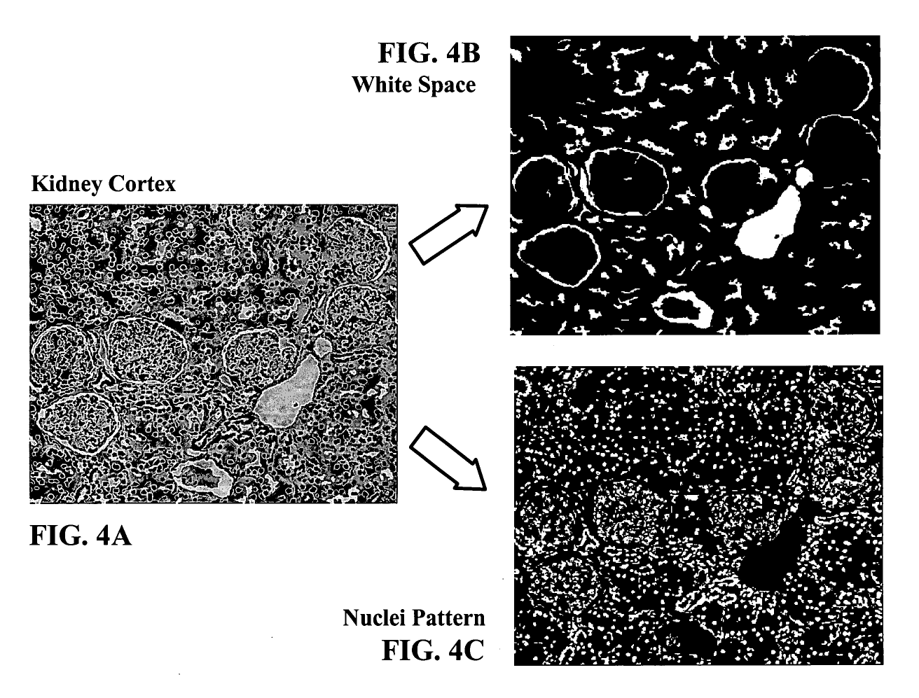

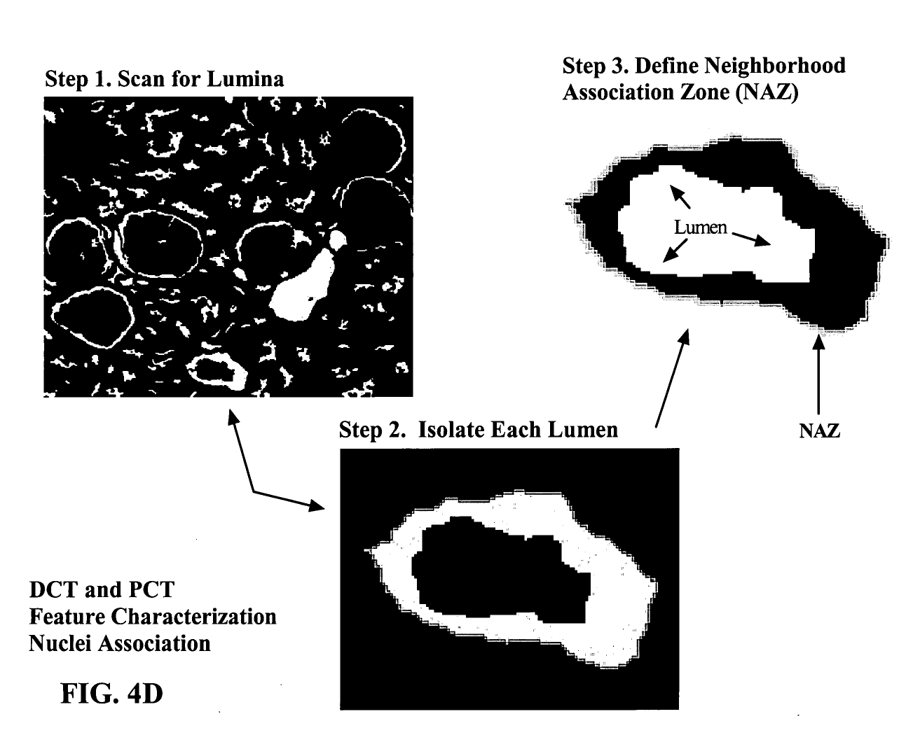

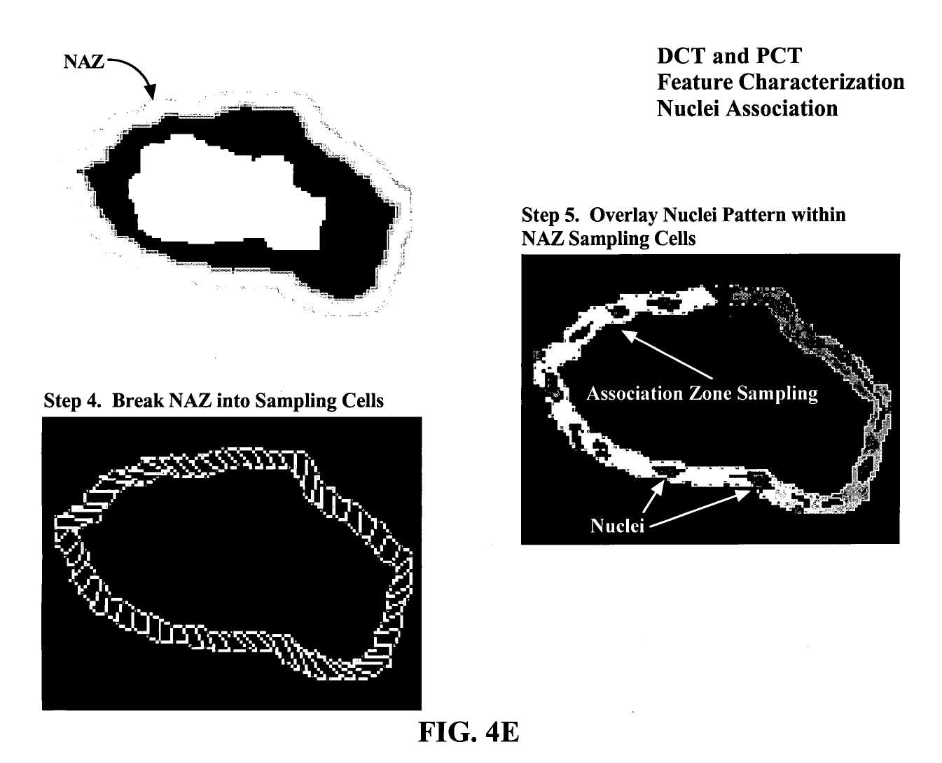

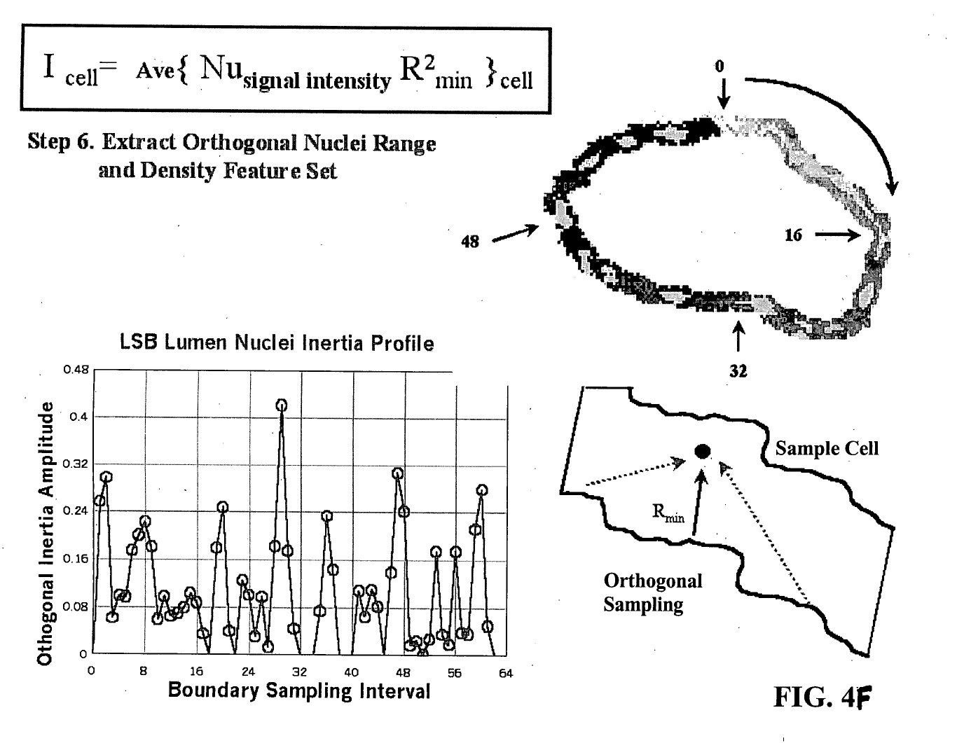

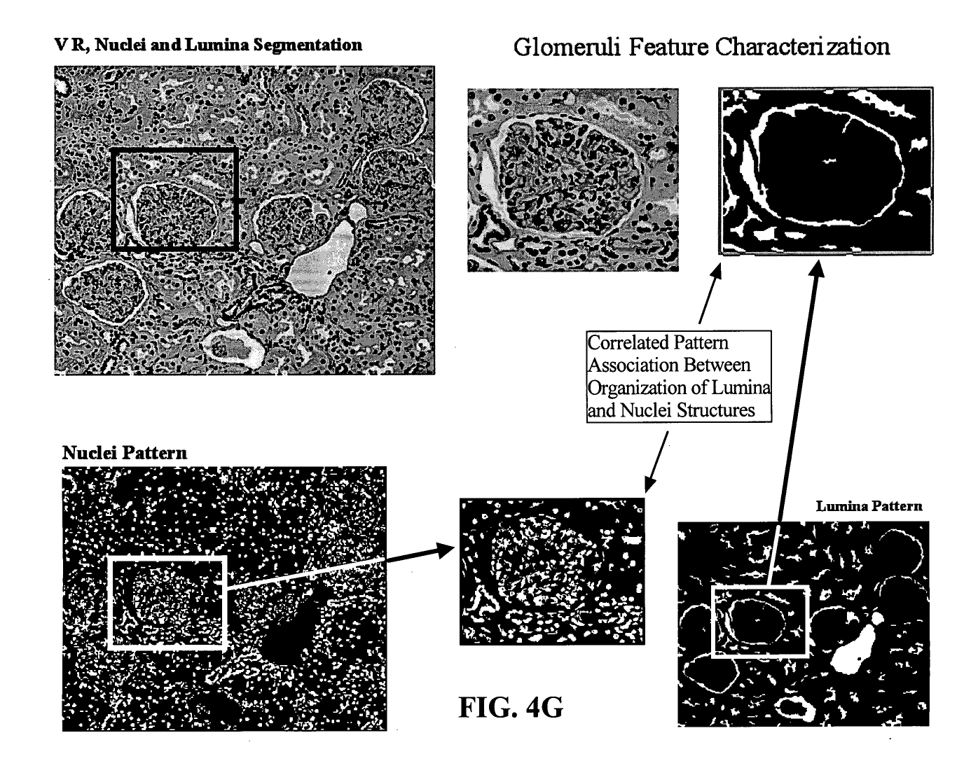

Three structures are important in the Kidney Cortex: glomeruli, proximal convoluted tubules (PCTs) and distal convoluted tubules (DCTs). glomeruli and DCTs can currently be recognized. FIGS. 4A-G illustrate aspects of FilterKidneyCortexMap discussed below.

Glomeruli appear in the Kidney Cortex as rounded structures surrounded by narrow Bowman's spaces. Recognition of a glomerulus requires location of the lumen that makes up the Bowman's space along with recognition of specific characteristics of the size and shape of the entire structure. In addition the quantification of CD31 (Vector red) staining information can be used because glomeruli contain capillaries and endothelial cells that typically stain VR-positive.

The bulk of the parenchyma tissue between each glomerulus consists of tubules that differ from one another in diameter, size, shape and staining intensity. The tubules mainly consist of proximal convoluted tubules with a smaller number of distal convoluted tubules and collecting ducts. A DCT may be differentiated from PCT by a larger more clearly defined lumen, more nuclei per cross-section, and smaller sections of length across the parenchyma tissue. The nuclei of the DCT lie close to the lumen and tend to bulge into the lumen.

The Kidney Cortex processing consists of the following steps:
1. Segmentation the white-space, nuclei, and Vector red regions. Each mask image is preprocessed by applying shape descriptors to eliminate regions that meet a certain criteria.
2. The white-space consists of lumen located around the glomeruli (Bowman's capsule), lumen located within tubular structures such as DCT and PCT, and areas within vessels and capillaries. The size, perimeter, distance to neighborhood objects, and density per neighborhood are used to select candidate lumen objects.
3. Nuclei density is used to further refine the list of candidate structures after lumen detection is complete. It is measured inside the Bowman's capsule and outside the perimeter of the tubular structures.
4. VR density, if CD31 staining is applied, is measured within the bowman's capsule and is used as the final discriminating factor for determining the existence of a glomerulus.

Glomeruli

Implementation: FilterGlomDetector

Glomeruli recognition requires four individual measurements on each candidate glomerulus. The lumen mask obtained in the segmentation process are preprocessed by eliminating small regions that are typically associated with blood vessels, small portions within tubular structures, and within glomeruli.
1. A compactness measurement is performed on each lumen object by measuring the ratio of the size of the lumen to the perimeter.
2. A Bowman's ring form factor measurement is obtained by measuring the ratio of the size of a circular ring placed around the lumen to the number of lumen pixels that intersect the bowman's ring. The size and diameter of the ring are based on computing bounding box measurements for the candidate lumen (e.g., width, height, and center coordinates). The ring is then rotated around the lumen and a form factor measurement is computed for each location. The location with the highest form factor measurement is kept.
3. The nuclei density of the ring is calculated as the ratio of nuclei pixels that intersect the form factor ring to the size of the ring.
4. Vector red density is calculated as the ratio of the Vector red pixels that intersect the form factor ring to the size of the ring.

A threshold is applied to each the compactness, form factor, nuclei density, and VR density measurements to determine if the candidate lumen is categorized as a glomerulus.

Distal Convoluted Tubules

Implementation: FilterTubeDetector

DCT recognition is accomplished by a method similar to epithelium detection. Each distal convoluted tubule (DCT), collecting duct (CD), and proximal convoluted tubule (PCT) can be modeled as a white space blob surrounded by a number of nuclei. The expected size of such a region to be related to a DCT is estimated empirically. Areas too large or too small are typically discarded during the glomeruli recognition process. Two methods can be used to complete the DCT recognition process:

Lumen Area to Nuclei Area Ratio

To decide if a lumen region is associated with a DCT, the nuclear content of an annular region about its boundary is examined. For an annular area to be classified as a DCT the nuclear content, e.g., the ratio of nuclear area to total area, must be very high. The decision threshold is again is determined empirically.

Lumen to Nuclei Distance Criterion

In this method, we compute the distance matrix between each white-space object and its neighboring nuclei (within certain radius). If the ratio between the areas of a white-space object and its neighboring nuclei is above a threshold and total area of the nuclei has is above a minimum requirement, they are classified as a DCT. This method can be used to identify PCTs by repeating the procedure with different parameters on the remaining white-space objects.

Kidney Medulla

Implementation: FilterDuctDetector, FilterKidneyMedullaMap

This algorithm is designed to detect and identify candidate collecting ducts in the kidney medulla. It is completed in three major parts: image layer segmentation, shape filters applied to measure candidate object properties, and finally a analysis test to identify the ducts. The segmentation involves white-space detection with removal of small areas and then nuclei detection. Distance filters are applied to compute the distance between candidate lumen and the closest surrounding nuclei. The final analysis identifies ducts that match specific criteria for distance between nuclei and lumen and nuclei to lumen ratio.

Liver

Implementation: FilterLiverMap

The goal of the liver algorithm is to delineate those areas that correspond to ducts and secondly those areas that comprise a portal triad. Ducts can be determined from a “good” nuclei image. The (boundary of) ducts correspond to the large components in the nuclei image. Throwing away very elongated components filters the set of large components. The remaining components are deemed to be ducts.

A portal triad consists of a vein, an artery and a duct, though often the artery is not clear. Since one does not generally expect to find nuclei in either the vein or artery, the algorithm finds areas of the appropriate size without nuclei that are near ducts found previously. These nuclear free areas are estimated in two ways. The brightness segmentation algorithm (see FilterSegment), produces a white space image. The image is filtered for areas of the appropriate size and shape to be arteries or veins. Nuclei-free areas are also estimated in a manner analogous to that discussed for finding glands in the prostate (see Prostate—Glands). Each of the areas (the duct-area and the nuclei-free area), which are disjoint, are then expanded. The intersection of the expanded regions is taken as the center of a ROI for a portal triad.

Lung

Implementation: FilterLungMap

Alveoli

Alveoli detection is done by morphological filtering of the tissue mask (see FilterTissueMask). The goal of the algorithm is to filter the tissue mask so that only tissue with a web-like shape remains after processing. The steps are as follows:
1. The image is initially down-sampled to an effective magnification of 2.5× in order to maximize execution speed.
2. The tissue mask is calculated using the Texture-Brightness method.
3. A median filter is applied to suppress the noise.
4. The image is inverted and a morphological close operation is performed using a disk structural element. This removes the alveolar tissue from the image.
5. Remaining islands of tissue are removed by size filtering.
6. A guard band is placed around remaining tissue areas by dilation an a second size filter is applied.
7. The resulting mask is combined with the initial tissue mask, producing the alveoli tissue.
8. The image is re-sampled to its original size.

Respiratory Epithelium

Respiratory epithelium is detected by applying a “double threshold” to the nuclei density and filtering out areas of the wrong shape. The steps are as follows:
1. The nuclei mask is computed (see FilterNuclei) and intersected with the complement of the alveoli mask. This reduces the search for epithelium to non-alveolar tissue.
2. The nuclei density map is computed using an averaging filter, and a threshold is applied to segment the areas with higher density. Because the nuclei segmentation can occasionally misestimate the nuclei, the threshold is determined relative to the trimmed mean of the density. The procedure is then repeated on the resulting mask (using a fixed threshold) to find areas that have high concentration of high nuclei density areas. These are the potential epithelial areas.
3. A morphological close operation is applied to join potential epithelial areas.
4. A shape filter is applied to remove areas that are outside of the desired size range, or that are too rounded. A more stringent shape criterion is used for areas that are closer to the top of the size range.
   Placenta

Implementation: FilterPlacenta
Generic tissue location is used for selecting regions of interest in Placenta since no specific structures need to be found. The basic concept is to identify the regions of the image where tissue is present (i.e. avoid large areas of white space). Therefore, the algorithm consists of two steps:
   1. The Vector red signature is suppressed to avoid false alarms in case of non-specific antibody binding to the glass.
   2. The white-space mask is computed using the Texture-Brightness method and the mask is inverted (positive becomes negative and negative becomes positive).
   3. A median filter is applied to “smooth out” noisy areas such as tiny white-space holes in tissue and small specks of material in the white-space. This has the effect of improving the quality of the ROI selection results. The size of the median filter window is proportional to the image magnification, where the proportionality constant can be adjusted per tissue.
      Prostate

Implementation: FilterProstateMap

Two structures are recognized in Prostate: glands and stroma.

Glands

Glands are recognized by the epithelium ring that surrounds them. The procedure for gland detection involves a two-step process where a sequence of morphological operations is followed by a sequence of tests. For the first step, we start with the nuclei mask and obtain candidate gland regions by the following sequence of algorithmic operations:
1. The nuclei are expanded using the procedure discussed above. This has the effect of connecting nuclei that are close together.
2. A clean-up algorithm is run to remove expanded nuclei that are not epithelial. This involves an iterative series of tests where isolated nuclei are progressively removed.
3. Small components are removed by connected component labeling and the resulting image is inverted (i.e. pixels that are ON are turned OFF and pixels that are OFF are turned ON).
4. Using a disk-shaped structural element, a morphological opening operation is performed. This results in a binary mask where candidate gland areas are marked.
5. Remaining holes in the candidate gland areas are filled in, resulting in a number of “morphological components”.

The second step consists of labeling the morphological components and performing two tests. For each labeled component, we compute:
1. The ratio of the component's area (in pixels) to the portion of its area occupied by nuclei.
2. The ratio of the component's area to the portion of its area which is occupied by pixels that have the middle gray-level value resulting from the brightness segmentation of the original image.

If the ratios are both greater than their respective thresholds, the component is labeled a gland.

Stroma

Stroma detection is performed by expanding the detected glands to account for the epithelial cells and inverting the image. In principle this produces a stroma mask. However, if the above algorithm misses a gland, then the area will incorrectly be labeled as stroma and can cause the ROI selector to locate a stroma ROI in a gland. To alleviate this problem, the initial stroma mask is intersected with the complement of the white-space (the tissue mask). This removes the white-space and the interior of any missed glands.

Testis

Implementation: FilterTestisMap

This algorithm is designed to map the interstitial region and Leydig cells of the testis. The initial step is to segment the image into nuclei and white-space/tissue layer images. Next the nuclei density is computed from the nuclei image and then thresholded. The initial interstitial region is found by taking the “exclusive OR” (or the absolute difference) of the tissue/white-space image and the nuclei density image.

The candidate Leydig cell regions are found by taking the product of the original image and the interstitial region. The candidate Leydig cells are found by taking the product of the previous Leydig cell region image and the nuclei density image. The final cells are identified by thresholding using a size criterion.

Thymus

Implementation: FilterThymusMap

The relevant features to be recognized in the Thymus are lymphocytes and Hassall's corpuscles. Direct recognition of these features at low magnification is not feasible. However, both can be found indirectly by using other information.

Lymphocytes

Lymphocytes are found at high concentration in the cortex region of the thymus. Therefore, an algorithm that identifies the cortex will also mark the lymphocytes with high probability. The cortex is recognized by its high nuclei density. A threshold is applied to the density map to obtain the high-density areas, followed by median filtering to remove noise and a morphological dilation step to improve coverage and join regions that re close together. Although this method does not insure 100% coverage, it consistently marks sufficient cortex area to locate lymphocytes.

Hassall's Corpuscles

A map of potential corpuscles (for the purpose of ROI selection) can be obtained by finding “gaps” in the thymus medulla, followed by the application of tests to eliminate objects that are not likely to be corpuscles. Potential corpuscles are regions of low nuclei density that is not white-space and is surrounded by medulla (a region of medium nuclei density). Size and shape filtering is done to reduce false alarms. The algorithm steps are:
1. Find the areas of low nuclei density by thresholding the nuclei density map and apply a median filter to reduce noise.
2. Find the tissue areas (see FilterTissueMask) and apply a median filter to reduce noise.
3. Intersect low nuclei density areas with the tissue mask to get a first pass at Hassall's corpuscles.
4. Union first pass Hassall's corpuscles with cortex. This helps avoid blobs that are connected to the cortex by making them bigger so they can be filtered out by size.
5. Filter out objects of the wrong size/shape combination.
6. Remove objects whose perimeter is not bordered by a sufficient number of medulla pixels.
   Thyroid

Implementation: FilterThyroidMap, FilterThyroidZone

The single structure of interest in the Thyroid is the follicles. This algorithm maps the follicular cells in the Thyroid by selecting nuclei structures that surround areas which are devoid of nuclei and are within the proper size and shape range. This is accomplished in the following steps:
1. The nuclei mask is obtained (see FilterNuclei).
2. The nuclei are joined with an algorithm that connects components which are close together using the “line” method (see FilterJoinComponents). The result is to isolate areas that are either white-space or the interior of follicles.
3. The image is inverted and morphologically opened with a large structural element to separate the individual objects. Resulting objects are either follicles or white-space.
4. A shape filter is applied in order to remove objects that are not sufficiently rounded to be “normal” looking follicles.
5. A guard band is created around the remaining objects using a morphological dilation operation, and the resulting image is combined with the previous by an exclusive union operation (XOR). This results in rings that mark the location of the follicular cells.
   Uterus

Implementation: FilterUterusZone

Three structures are recognized in the uterus: stroma, smooth muscle and glands. The algorithm first segments the input image into nuclei, cytoplasm, and white space. Based on the segmentation result, the “density map” for each class is calculated.

Stroma

To determine the potential stroma regions, apply the Otsu threshold to segment the nuclei density map. Regions where nuclei density exceeds the Otsu threshold value are labeled as potential stroma regions. A size based filter is then used to clean up the spurious stroma regions. To fill the holes within each blob in the potential stroma map, a morphological closing and a flood fill operations are applied.

Smooth Muscle

To determine the potential muscle regions, we find all the regions not labeled as stroma and where the nuclei density map exceeds an empirically determined threshold value.

Glands

To find the glands, we follow the following steps:
1. Find the areas where the nuclei density is below an is empirically determined threshold value. A size based filter is used to clean up the resulting areas.
2. Filter out the potential glands not intersecting with the stroma regions. Each blob representing a potential gland can now be considered as a seed for a region growing operation.
3. Repeatedly perform a special seeded region growing operation that only allows region growing as long as the growth does not over any nuclei regions. The number of times of the seeded region growing operation is repeated is empirically determined. Apply a size based filter to clean up the spurious glands, followed by morphological dilation and flood fill operations to remove the holes in the glands and make them a bit “fatter” for further analysis.
4. Label the epithelium surrounding the potential glands by segmenting the nuclei density map and retaining the areas where the nuclei density exceeds a threshold (that threshold value is empirically determined). This produces a map in which only the pixels on the epitheliums next to glands are “on”. The blobs in the map are dilated so that they will partially overlap with the potential glands estimated previously.
5. For each potential gland, calculate the perimeter (p) and the fraction r of p that overlaps with the map from the previous step. Apply a threshold on p and retain the glands whose r-value exceed the given threshold value. The consequence of this sequence of operations is to remove the potential glands that do not have a sufficient amount of epithelium surrounding them. This removes the vessels that could be mistaken as glands.
   End of Table 5.

Continuing the example of determining the presence of a structure of interest for the colon 162 tissue type, Table 5 provides additional discussion how the structure-identification algorithm FilterColonZone correlates cellular patterns of colon tissue to determine the presence of one or more of epithelium, smooth muscle, submucosa, and muscularis mucosa tissue constituents.

Table 6 provides yet further discussion of several filters of the filter class 180 for the extraction or segmentation of basic tissue constituent features.

### Table 6

Brightness Image Segmentation

Implementation: Segment, FilterSegment

This basic algorithm produces a three-level grayscale image from the source RGB image. For many vision tasks it is appropriate to reduce the data to a binary image. The natural reduction in histological images is a ternary image as there are 3 fundamental areas, generally corresponding (in order of brightness) to: nuclei, cytoplasm, and white space areas. The RGB image is first converted to grayscale by taking the root mean square (RMS) average image. The next step is to sort the all the gray level values (darker values are lower brighter values are higher). Finally, the values are clustered as follows: Let C₁ be the average of the darkest D % of the pixels (where D is typically 20), C₂ the average of the “middle” 45%-55%, and C₃ be the average of the top T % of the sorted values (where T is typically 10). A pixel is then placed in one of 3 groups depending on which C₁ is nearest its gray value. The regions 0-20%, 45-55% and 90-100% above work well for many tissues, and can be adaptively (empirically) chosen on a per tissue basis. Due to illumination variation, especially “barrel distortion”, the procedure above sometimes produces better results if done locally. To do so, a small window is chosen so that the illumination within it is uniform and this window is moved across the entire image.

Two variations on the algorithm have been implemented to accommodate image variability. In the first, C₁ is taken to be the average of the bottom 2%, C₃ is the average of the top 2% and C₂ is the sum:
C₂=tC₁+(1−t)C₃.

The scheme above is applied with these values to produce a tri-color image. The value t=0.15 has worked quite well for many tissues. Unfortunately, this method tacitly assumes that the at least the top 2% of the gray level values are indeed white space. This assumption occasionally fails (e.g., in some liver images). A second variation tries to estimate the region of the in the gray levels that corresponds to white space. The criterion used is that the white space is very flat or has very little texture. Hence (in small patches) the variation in the gray values from the mean is close to zero. In this case, C₃ is taken as the average of those gray values whose associated pixel has a small standard deviation in a window about it. Both C₁ and C₂ are chosen as before.

Nuclei Segmentation

Brightness-Color Method

Implementation: FilterNuclei

Two approaches to nuclei segmentation have been developed. In the first, nuclei are segmented (their corresponding pixels are labeled) by a combination of brightness and color information. Two binary masks are computed and combined using a logical AND Boolean operation to produce the best possible nuclei mask. The first mask is obtained from the dark (lowest brightness level) pixels produced by the brightness segmentation discussed above. The second mask is obtained by performing the following test on each pixel in the image:

$$B - \frac{G + R}{2} > threshold$$

where B, G and R are the brightness levels for blue, green and red respectively in the RGB image.

White-Space Segmentation

Two methods for white-space segmentation are discussed. The first is based on the brightness image segmentation. The second uses a texture map in combination with brightness.

Brightness Method

Implementation: Segment, FilterWhiteSpace

This method simply extracts the top brightness level from the result of the brightness segmentation algorithm discussed above. This approach works well in tissue types that tend to have small amounts of white-space uniformly distributed through the image, such as Heart and Skeletal Muscle.

Texture-Brightness Method

Implementation: FilterTextureMap, FilterTissueMask, FilterWhiteSpace

The premise of this method is that tissue and non-tissue areas can be separated by a combination of their texture and brightness. The white-space is obtained by the combination of two binary mask images. The first mask results from the application of a texture threshold. Using the first mask, a brightness threshold is computed and applied to obtain a second mask that is then combined with the first.

To obtain the first (texture-based) mask, a texture map is computed from the brightness image using a variance-based measure (see FilterTextureMap). Application of a threshold (typically in the range of 0.5 to 1.0, but varying depending on pre-processing steps) to the texture map results in a binary image where the high-texture regions are positive and the low-texture regions negative. Taking only the negative (low-texture) pixels from the texture image, one can compute a brightness threshold using a simple statistic (the mean brightness minus twice the standard deviation). Application of this threshold produces a second binary image where the brighter regions are positive and the darker ones negative. Finally the white space mask is obtained by creating a third binary mask where a pixel is positive (white-space) if the corresponding pixel from the texture-based mask is negative or if the corresponding pixel from the brightness-based mask is positive.

This approach works well in cases where there the tissue texture is distinct from that of the white-space. Such tissue types include Colon, Spleen and Lung.

Vector Red Segmentation

Implementation: Segment

Although Vector red (VR) cannot be assumed to be consistently present in most tissues, there are cases where the antibody that is tagged with VR will always bind to certain kinds of cells such as vessel endothelium and glomerulus cells, and so can be used to improve the results from structure recognition by other means. For many images, VR segmentation can be achieved by a comparison between the brightness of the red channel relative to the blue channel. If the red channel value for a given pixel is greater than the corresponding blue channel value by a set margin, then the pixel is marked as VR.

Vector Red Suppression

Implementation: FilterSuppressVR

High levels of Vector red (VR) in a sample can significantly affect the performance of feature extraction algorithms. Using a remote spectroscopy technique known as unconstrained linear unmixing, the VR signature in the image can be digitally suppressed to any extent desired. Linear unmixing begins with the assumption that a pixel's spectral optical depth is the result of a linear mixing process:
d=Ma+e,
where d is the vector of pixel optical depths for red, green and blue, a is the vector of relative abundances of the pixel's components, e is the model error, and M is a matrix whose columns contain the component's normalized color signatures. The optical depth values are obtained by the formula:
d=−log(g+0.1/255.1),
where g is the gray level value triple (R, G, B) for each pixel. The value of 0.1 is used to avoid computing the logarithm of zero while introducing negligible distortion. Currently, the components used in the mixture model are VR, hematoxilin and white-space. The latter is represented by a “gray” signature where all the colors have equal weight. To suppress the VR signature, we estimate a using the pseudo-inverse method:
â=(M^{T}M)⁻¹M^{T}d,
and multiply the element of â corresponding to VR by a factor in the range [0,1], where a value of 0 corresponds to complete VR suppression and a value of 1 corresponds to no suppression. Finally, a new optical depth vector is obtained by application of the first equation to the new abundance vector, and the VR-suppressed RGB image is re-formed. This algorithm assumes that a pixel's color is derived only from the components in the model. Other colors/stains will produce unpredictable results, but the effect will be confined to the affected pixels only.
End of Table 6.

Table 7 provides additional discussion of alternative embodiments of the tissue mapping tools of the filter class 180 that generate maps of statistics measured from segmented images.

### Table 7

Nuclei Mapping

One informative measure is the density of nuclei at a particular point in the image. Two types of nuclei density measures have been developed: a simple linear density and a fractal density.

Linear Density

Implementation: FilterFastAverage

The linear density is computed by convolving the binary nuclei mask with an averaging window of a given size. This provides a measure at each pixel of the average fraction of image area that is designated as nuclei. Such information is useful in mapping zones within tissues such as Thymus.

Fractal Density

Implementation: FilterFractalDensity

Fractal descriptors measure the complexity of self-similar structures across different scales. The fractal density (FD) mapping measures the local non-uniformity of nuclei distribution and is often termed the fractal dimension. One method for implementing the FD is the box-counting approach. We implement one variation of this approach by partitioning the image into square boxes of size L×L and counting the number of N(L) of boxes containing at least one pixel that is labeled as nuclear. The FD can be calculated as the absolute value of the slope of the line interpolated to a log(N(L))×log(L) plot. The sequence of box sizes, starting from a given size L over a given pattern in the image, is usually reduced by ½ from one level to the next. FD measurements 2>FD>1 typically correspond to the most fractal regions, implying more complex shape information.

Other Tools

Nuclei Expansion and Contraction

Implementation: FilterExpandNuclei, FilterErodeNuclei

Expanding and contracting the nuclei is useful in the process of computing some structure masks. Although similar in concept to morphological dilation and erosion, the implementation is significantly different. In the expansion operation, the neighborhood of adjoining pixels around each pixel in a mask is checked for the presence of pixels that are positive (non-zero). If the number of non-zero pixels exceeds a threshold, then all the pixels in the neighborhood are turned “on” (i.e. given a non-zero value). Otherwise they are turned “off” (i.e. set to zero). Note that, depending on the threshold, small, isolated areas can be removed by the algorithm, providing a filtering capability.

The contraction operation follows the same basic procedure as the expansion, but the neighborhood pixels are turned “off” if the number of non-zero pixels is less than the given threshold.

Object Joining

Implementation: FilterJoinComponents

One of two methods is used to fill in the background between objects that are close together. In both cases a window size is specified and the window is places over each pixel in the input image. In the Line method, if the center pixel has a non-zero value then it is joined to each non-zero pixel in that window by the straight-line segment connecting the two pixels (i.e., each pixel on the connecting line is turned on). In the Center-of-Mass method, the center of mass of the pixels is computed and turned on.

Region of Interest Selection

Implementation: FilterROISelector

A step is the selection of Regions of Interest (ROIs) from low-magnification images in order to provide the system with the locations within the tissue where a high-magnification image should be collected for further analysis. Interesting regions in tissues are those associated with structures of interest.

The ROI selection process begins with a binary mask that has been computed to mark the locations of a particular structure type, such as glands, ducts, etc., in the tissue section. The algorithms used to create such masks are discussed in elsewhere in this document. Given the desired number of ROIs, the mask image is divided into a grater number of approximately equal size sections. For each section, an optimal location is selected for the center of a candidate ROI. Each candidate ROI is then “scored” by computing the fraction of pixels within the ROI where the mask has a positive value, indicating to what extent the desired structure is present. The ROIs are then sorted by score with an overlap constraint, and the top-scoring ROIs are selected.

To select the optimal locations within each image section, a multi-resolution method is used where the image section if further sub-divided in successive steps. At each step a “best” subsection is selected and the process is repeated until the subsections are pixel-sized. This method does not insure a globally optimum location will be selected each time, but does consistently produce good results. Selection of a “best” subsection at each step requires that a Figure of Merit (FOM) be computed for each subsection at each step. A FOM is a value that indicates the “goodness” of something, with a higher number always being better than a lower number. For tissue ROI selection, a reasonable FOM is obtained by filtering the binary mask with an averaging window of size matching the ROI. The resulting FOM image is not binary, but rather has values that range from 0 to 1, depending on the proportion of positive mask pixels within the averaging window. To obtain a FOM for a given subsection, the FOM image is simply averaged over all the pixels in the subsection. Although seemingly redundant, this procedure insures that ROI selections will be centered in areas with the broadest possible mask coverage.

End of Table 7.

The utility class 170 of FIG. 2 includes general tools. A portion of the utility subclasses of the utility class 170 is illustrated in FIG. 2 as CBlob 171, CLogical 172, and CMorph 173. Table 8 discusses several utility subclasses of the utility class 170.

TABLE 8

| Class | Description |
|---|---|
| CBlob | Connected component labeling and associated functions |
| | (flood fill, size filtering and shape filtering) |
| CElemOps | Elementary functions that operate on arrays |
| (template) | |
| CEntropy | Functions to find optimal threshold by the relative |
| | entropy criterion |
| CKidneyUtil | Set of utility functions for processing Kidney images. |
| CLogical | Logical functions (and, or, xor, max, min, minus) |
| CLUT | Implements a look-up table |
| (template) | |
| CMorph | Binary morphology operations with different structural |
| | elements (dilate, erode, open, close, invert) |
| CMosaic | Image tile stitching program |
| COtsu | Functions to find optimal threshold by the Otsu criterion |
| CPCA | Functions to perform Principal Component Analysis and |
| (template) | related procedures |
| CPlane | Functions to manipulate the planes of an RGB image |
| CRegistryKey | Functions to access the Windows registry |
| CSegment | Functions for image segmentation |
| CZoneUtil | Functions supporting zone mapping filters (Filter * Zone) |

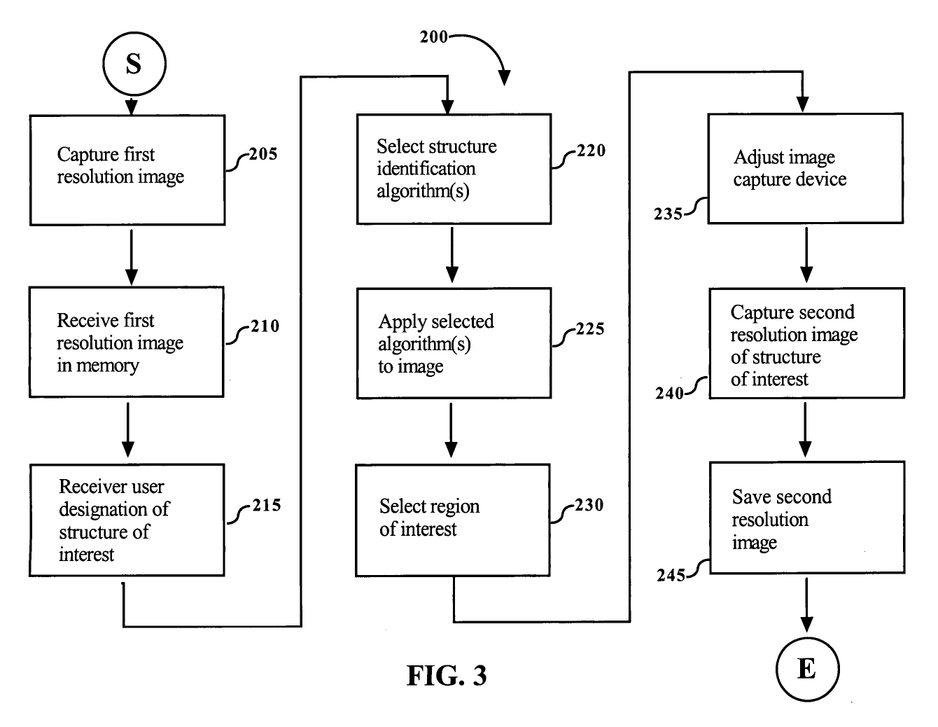

FIG. 3 is a diagram illustrating a logical flow 200 of a computerized method of automatically capturing an image of a structure of interest in a tissue sample, according to an embodiment of the invention. The tissue samples typically have been stained before starting the logical flow 200. The tissue samples are stained with a nuclear contrast stain for visualizing cell nuclei, such as Hematoxilin, a purple-blue basic dye with a strong affinity for DNA/RNA-containing structures. The tissue samples may have also been stained with a red alkaline phosphatase substrate, commonly known as “fast red” stain, such as Vector® red (VR) from Vector Laboratories. Fast red stains precipitate near known antibodies to visualize where the protein of interest is expressed. Such areas in the tissue are sometimes called “Vector red positive” or “fast red positive” areas. The fast red signal intensity at a location is indicative of the amount of probe binding at that location. The tissue samples often have been stained with fast red for uses of the tissue sample other than determining a presence of a structure of interest, and the fast red signature is usually suppressed by structure-identification algorithms of the invention.

After a start block S, the logical flow moves to block 205, where a microscopic image of the tissue sample 26 at a first resolution is captured. Also at block 205, a first pixel data set representing the captured-color image of the tissue sample at the first resolution is generated. Further, the block 205 may include adjusting an image-capture device to capture the first pixel data set at the first resolution.

The logic flow moves to block 210, where the first pixel data set and an identification of a tissue type of the tissue sample are received into a memory of a computing device, such as the memory 104 of the computing device 100. The logical flow then moves to block 215 where a user designation of a structure of interest is received. For example, a user may be interested in epithelium tissue constituents of colon tissue. At block 215, the logic flow would receive the user's designation that epithelium is the structure of interest.

Next, the logic flow moves to block 220, where at least one structure-identification algorithm responsive to the tissue type is selected from a plurality of stored structure-identification algorithms in the computing device. At least two of the structure-identification algorithms of the plurality of algorithms are responsive to different tissue types, and each structure-identification algorithm correlating at least one cellular pattern in a given tissue type with a presence of a structure of interest for the given tissue type. The structure-identification algorithms may be any type of algorithm that can be run on a computer system for filtering data, such as the filter class 180 of FIG. 2.

The logical flow moves next to block 225, where the selected at least one structure-identification algorithm is applied to the first pixel data set representing the image. Using the previous example where the tissue type is colon tissue, the applied structure-identification algorithm is FilterColonZone. Tables 3 and 5 describe aspects of this filter as segmenting the first pixel data set into three classes of regions: nuclei, cytoplasm, and white space. Based on the segmentation result, a “density map” for each class is calculated. Using the density maps, the algorithm finds the potential locations of the “target zones” or cellular constituents of interest: epithelium, smooth muscle, submucosa, and muscularis mucosa. Each potential target zone is then analyzed with tools for local statistics, and morphological operations performed in order to get a more precise estimation of its location and boundary. Regions in an intermediate mask are labeled with the following gray levels for the four cellular constituents: epithelium—50, smooth muscle—100, submucosa—150, and muscularis Mucosa—200.

To obtain the epithelium regions, the Otsu threshold technique is applied to the nuclei density map. The regions where the nuclei density exceeds the Otsu threshold value are classified as potential epithelium. Among the potential epithelium regions, an “isolated blob removal” process is applied, which removes the isolated blobs for a given range of sizes, and within certain ranges of “empty” neighborhood. The next step is to invoke a shape filter that removes the blobs that are too “elongated” based on the eigen-axes of their shapes. A morphological dilation then smoothes the edges of the remaining blobs. The result of this sequence of operations is a set of pixels that correlates closely with the epithelium regions.

To find the submucosa regions, a variance map of the gray-scale copy of the original image is first produced. The Otsu threshold is then applied to the variance map. It segments out the potential submucosa and epithelium regions by retaining the portion of the variance map where the variance exceeds the Otsu threshold values. Since the submucosa regions are disjointed with the epithelium, the latter can be removed, and a potential submucosa map is thus produced. A size-based filter is then applied to remove blobs under or exceeding certain ranges. A set of pixels that correlates closely with the sub-mucosa regions is thus obtained.

To find the potential muscle regions, the Otsu threshold is applied to the cytoplasm density map. The regions of the map where the density values exceed the threshold value are labeled as the initial estimate for potential muscle regions. After excluding the epithelium and submucosa regions from the potential muscle regions, an isolated blob remover is used to filter out the blobs that are too large or too small and with sufficiently “empty” neighbor regions. This sequence of operations results in a set of pixels that correlates closely with the final muscle map.

A binary structure mask is computed from the filter intermediate mask generated by the structure-identification algorithm(s) applied to the first pixel data set. The binary structure mask is a binary image where a pixel value is greater than zero if a pixel lies within the structure of interest, and zero otherwise. If the filter intermediate mask includes a map of the user-designated structure of interest, the binary structure mask may be directly generated from the filter intermediate mask. If the filter intermediate mask includes cellular components requiring correlating to determine the presence of the structure of interest, the cellular components, a co-location operator is applied to the intermediate mask to determine whether there is a coincidence, an intersection, a proximity, or the like, between the cellular components of the intermediate mask. By way of further example, if the designated structure of interest for a colon tissue sample had included all four tissue constituents listed in Table 1, the binary structure mask will describe and determine a presence of a structure of interest by the intersection or coincidence of the locations of the cellular patterns of at least one of the four constituents constituting the structure of interest.

The binary structure mask typically will contain a “1” for those pixels in the first data sets where the cellular patterns coincide or intersect and a “0” for the other pixels. When a minimum number of pixels in the binary structure mask contain a “1,” a structure of interest is determined to exist. If there are no areas of intersection or coincidence, no structure of interest is present and the logical flow moves to an end block E. Otherwise, the logical flow moves to block 230 where at least one region of interest (ROI) having a structure of interest is selected for capture of the second resolution image.

A filter, such as the FilterROISelector discussed in Tables 2, 4, and 7, uses the binary structure mask generated at block 225 marking locations of the cellular constituents comprising the structure of interest to determine a region of interest. A region of interest is a location in the tissue sample for capturing a second resolution image of the structure of interest. A method of generating a region of interest mask includes dividing the binary structure mask image into a number of approximately equal size sections greater in number than a predetermined number of regions of interest to define candidate regions of interest. Next, an optimal location for a center for each candidate region of interest is selected. Then, each candidate region of interest is scored by computing the fraction of pixels within the region of interest where the mask has a positive value, indicating to what extent the desired structure is present. Next, the candidate regions of interest are sorted by the score with an overlap constraint. Then, the top-scoring candidate regions of interest are selected as the regions of interest.

Selecting the region of interest at block 230 may also include selecting optimal locations within each region of interest for capture of the second pixel data set in response to the figure-of-merit process discussed in Tables 3 and/or 7 above. A method of selecting optimal locations in response to a figure-of-merit includes dividing each region of interest into a plurality of subsections. Next, a “best” subsection is selected by computing a figure of merit for each subsection. The figure of merit is computed filtering the binary structure mask with an averaging window of size matching the region of interest for a resulting figure of merit image that has values ranging from 0 to 1, depending on the proportion of positive mask pixels within the averaging window; and obtaining a figure of merit for a given subsection by averaging the figure of merit image over all the pixels in the subsection, with a higher number being better than a lower number. Finally, repeating the dividing and selecting steps until the subsections are pixel-sized.

The logic flow then moves to block 235, where the image-capture device is adjusted to capture a second pixel data set at a second resolution. The image-capture device may be the robotic microscope 21 of FIG. 1. The adjusting step may include moving the tissue sample relative to the image-capture device and into an alignment for capturing the second pixel data set. The adjusting step may include changing a lens magnification of the image-capture device to provide the second resolution. The adjusting step may further include changing a pixel density of the image-capture device to provide the second resolution.

The logic flow moves to block 240, where the image-capture device captures the second pixel data set in color at the second resolution. If a plurality of regions of interest are selected, the logic flow repeats blocks 235 and 240 to adjust the image-capture device and capture a second pixel data set for each region of interest. The logic flow moves to block 245 where the second pixel data set may be saved in a storage device, such in a computer memory or hard drive. Alternatively, the second pixel data set may be saved on a tangible visual medium, such as by printing on paper or exposure to photograph film.

The logic flow 200 may be repeated until a second pixel data set is captured for each tissue sample on a microscope slide. After capture of the second pixel data set, the logic flow moves to the end block E.

An alternative embodiment, the logic flow 200 includes an iterative process to capture the second pixel data set for situations where a structure-identification algorithm responsive to the tissue type cannot determine the presence of a structure of interest at the first resolution, but can determine a presence of regions in which the structure of interest might be located. In this alternative embodiment, at blocks 220, 225, and 230, a selected algorithm is applied to the first pixel data set and a region of interest is selected in which the structure of interest might be located. The image-capture device is adjusted at block 235 to capture an intermediate pixel data set at a resolution higher than the first resolution. The process returns to block 210 where the intermediate pixel data set is received into memory, and a selected algorithm is applied to the intermediate pixel data set to determine the presence of the structure of interest at block 225. This iterative process may be repeated as necessary to capture the second resolution image of a structure of interest. The iterative process of this alternative embodiment may be used in detecting Leydig cells or Hassall's corpuscles, which are often not discernable at the 5× magnification typically used for capture of the first resolution image. The intermediate pixel data set may be captured as 20× magnification, and a further pixel data set may be captured at 40× magnification for determination whether a structure of interest is present.

In some situations, an existing tissue image database may require winnowing for structures of interest, and possible discard of all or portions of images that do not include the structures of interest. An embodiment of the invention similar to the logic flow 200 provides a computerized method of automatically winnowing a pixel data set representing an image of a tissue sample having a structure of interest. The logical flow for winnowing a pixel data set includes receiving into a computer memory a pixel data set and an identification of a tissue type of the tissue sample similar to block 205. The logical flow would then move to blocks 220, 225, and 225 to determine a presence of the structure of interest in the tissue sample. Upon completion of block 230, the tissue image may be saved in block 245 in its entirety, or a location of the structure of interest within the tissue sample may be saved. The location may be a sub-set of the pixel data set representing the image that includes the structure of interest may be saved. The logic flow may include block 230 for selecting a region of interest, and sub-set of the pixel data set may be saved by saving a region of interest pixel data sub-set.

An embodiment of the invention was built to validate the method and apparatus of the invention for automatically determining a presence of cellular patterns, or substructures, that make up the structure of interest in a tissue sample for various tissue types. An application was written incorporating the embodiment of the invention discussed in conjunction with the above figures, and including the structure-identification algorithms of the filter class 180 of FIG. 2 as additionally discussed in Tables 2-7. The application was run on a computing device, and the validation testing results are contained in Table 8 as follows:

TABLE 8
Validation Testing Results

| Tissue | samples | Substructure | Success Rate |
|---|---|---|---|
| Bladder | 35 | Urothelium/Trans. Epith | 55% |
| | | Lamina Propria | 56% |
| | | Smooth Muscle | 75% |
| Breast | 36 | Ducts/Lobules | 63% |
| | | Stroma | 89% |
| Colon | 42 | Glands/Crypts | 67.90% |
| | | Muscularis Mucosa | 59.50% |
| | | Submucosa | 75.60% |
| | | Smooth Muscle | 86.90% |
| Kidney Cortex | 46 | Glomerulus/DCT | 95.70% |
| Kidney Medulla | 42 | Ducts | 98% |
| Liver | 36 | Portal Triad | 56% |
| Lung | 41 | Alveoli | 100.00% |
| | 48 | Respiratory Epithelium | 46.90% |
| Lymph Node | 18 | Lymphoid Follicle | 72% |
| Placenta | | | |
| Prostate | 40 | Glands | 98.80% |
| | | Stroma | 95.00% |
| Small Intestine | 24 | Glands/Crypts | 87.50% |
| | | Muscularis Mucosa | 60.40% |
| | | Smooth Muscle | 83.30% |
| | | Submucosa | 72.90% |
| Spleen | 42 | White Pulp | 76% |
| Stomach | 24 | Glands/Lam Prop | 80.20% |
| | | Muscularis Mucosa | 42.70% |
| | | Submucosa | 88.50% |
| | | Smooth Muscle | 83.30% |
| Thymus | | Lymphocytes | 100.00% |
| | | Hassall's Corpuscle | 57.50% |
| Tonsil | 40 | Lymphoid Follicle | 52% |
| Uterus | | Glands | 76.70% |
| | | Stroma | 65.30% |
| | | Myometrium | 76.10% |

The testing validated the structure-identification algorithms for the cellular components.

Certain aspects of the present invention are also discussed in the following United-States provisional patent applications, all of which are hereby incorporated by reference in their entirety. Application No. 60/265,438, entitled PPF Characteristic Tissue/Cell Pattern Features, filed Jan. 30, 2001; application No. 60/265,448, entitled TTFWT Characteristic Tissue/Cell Features, filed Jan. 30, 2001; application No. 60/265,449, entitled IDG Characteristic Tissue/Cell Transform Features, filed Jan. 30, 2001; application No. 60/265,450, entitled PPT Characteristic Tissue/Cell Point Projection Transform Features, filed Jan. 30, 2001; application No. 60/265,451, entitled SVA, Characteristic Signal Variance Features, filed Jan. 30, 2001; application No. 60/265,452, entitled RDPH Characteristic Tissue/Cell Features, filed Jan. 30, 2001; and application Ser. No. 10/120,206 entitled Computer Methods for Image Pattern Recognition in Organic Material, filed Apr. 9, 2002.

The various embodiments of the invention may be implemented as a sequence of computer-implemented steps or program modules running on a computing system and/or as interconnected-machine logic circuits or circuit modules within the computing system. The implementation is a matter of choice dependent on the performance requirements of the computing system implementing the invention. In light of this disclosure, it will be recognized that the functions and operation of the various embodiments disclosed may be implemented in software, in firmware, in special purpose digital logic, or any combination thereof without deviating from the spirit or scope of the present invention.

Although the present invention has been discussed in considerable detail with reference to certain preferred embodiments, other embodiments are possible. Therefore, the spirit or scope of the appended claims should not be limited to the discussion of the embodiments contained herein. It is intended that the invention resides in the claims hereinafter appended.

1. A computerized method of automatically capturing an image of a structure of interest in a tissue sample, comprising:
(a) receiving into a computer memory a first pixel data set representing an image of the tissue sample at a first resolution and an identification of a tissue type of the tissue sample;
(b) selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type;
(c) applying the selected at least one structure-identification algorithm to the first pixel data set to determine a presence of the structure of interest in the tissue sample; and
(d) capturing a second pixel data set at a second resolution, the second pixel data set representing an image of the structure of interest, the second resolution providing an increased degree to which closely spaced objects in the image can be distinguished from one another over the first resolution.

2. The method of claim 1, wherein each structure-identification algorithm further determines a location of the structure of interest within the tissue sample.

3. The method of claim 2, further including:
(e) selecting for inclusion within the second pixel data set at least one region of interest within the first pixel data set that includes the structure of interest.

4-10. (canceled)

11. A computerized method of automatically capturing an image of a structure of interest in a tissue sample, comprising:
(a) receiving into a computer memory a first pixel data set representing an image of the tissue sample at a first resolution and an identification of a tissue type of the tissue sample;
(b) selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type;
(c) applying the selected at least one structure-identification algorithm to the first pixel data set to determine a presence of the structure of interest in the tissue sample;
(d) adjusting an image-capture device to capture a second pixel data set at a second resolution, the second pixel data set representing an image of the structure of interest, the second resolution providing an increased degree to which closely spaced objects in the image can be distinguished from one another over the first resolution; and
(e) capturing a second pixel data set at a second resolution, the second pixel data set representing an image of the structure of interest, the second resolution providing an increased degree to which closely spaced objects in the image can be distinguished from one another over the first resolution.

12. The method of claim 11, wherein the tissue sample includes an animal tissue.

13. The method of claim 11, wherein the tissue sample includes cells in a fixed relationship.

14. The method of claim 11, wherein the cellular pattern is an intracellular pattern.

15. The method of claim 11, wherein the cellular pattern is an intercellular pattern.

16. The method of claim 11, wherein the adjusting step further includes changing a lens magnification to provide the second resolution.

17. The method of claim 11, wherein the adjusting step further includes changing a pixel density to provide the second resolution.

18. The method of claim 11, wherein the adjusting step further includes moving the image-capture device relative to the tissue sample.

19. The method of claim 11, wherein the capturing step further includes saving the second pixel data set in a storage device.

20. The method of claim 11, wherein the capturing step further includes saving the second pixel data set on a tangible visual medium.

21. The method of claim 11, wherein the capturing step further includes receiving the second pixel data set into the memory.

22. The method of claim 11, further including a step of adjusting the image-capture device to capture the first pixel data set at the first resolution.

23. The method of claim 11, wherein, if the applying step identifies a plurality of structures of interest, the applying step further includes a step of selecting at least one structure of interest over at least one other structure of interest.

24. The method of claim 23, wherein the capturing step further includes capturing the second pixel data set for each structure of interest having merit.

25. The method of claim 11, wherein the first pixel data set includes a color representation of the image.

26. A computer readable data carrier containing a computer program which, when run on a computer, causes the computer to perform the method of claim 11.

27. A computerized method of automatically winnowing a pixel data set representing an image of a tissue sample having a structure of interest, comprising:
(a) receiving into a computer memory the pixel data set and an identification of a tissue type of the tissue sample;
(b) selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type;
(c) applying the selected at least one structure-identification algorithm to the first pixel data set to determine a presence of the structure of interest in the tissue sample; and
(d) capturing a sub-set of the pixel data set that includes the structure of interest.

28. The method of claim 27, wherein the capturing step includes saving a location of the structure of interest within the image.

29. The method of claim 27, wherein the capturing step includes saving a region of interest within the image.

30. The method of claim 27, wherein the first pixel data set includes a color representation of the image.

31. A computer readable data carrier containing a computer program which, when run on a computer, causes the computer to perform the method of claim 27.

32. A computerized method of automatically determining a presence of a structure of interest in a tissue sample, comprising:
(a) receiving into a computer memory a first pixel data set representing an image of the tissue sample at a first resolution and an identification of a tissue type of the tissue sample;
(b) selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type; and
applying the selected at least one structure-identification algorithm to the first pixel data set to determine a presence of the structure of interest in the tissue sample.

33-87. (canceled)

88. A computerized image capture system, the system comprising:
(a) a controllable image-capture device operable to capture digital images of a tissue sample; and
(b) a computer operable to control the image-capture device and receive the captured digital images of tissue sample, the computer including a memory, a storage, a processor, and an image capture application;
(c) the image capture application including computer executable instructions that automatically capture an image of a structure of interest in a tissue sample, the instructions including the steps of:
(i) receiving into the computer memory a first pixel data set representing an image of the tissue sample at a first resolution and an identification of a tissue type of the tissue sample;
(ii) selecting at least one structure-identification algorithm responsive to the tissue type from a plurality of structure-identification algorithms, at least two of the structure-identification algorithms of the plurality of algorithms being responsive to different tissue types, and each structure-identification algorithm correlating at least one cellular pattern in a given tissue type with a presence of a structure of interest for the given tissue type;
(iii) applying the selected at least one structure-identification algorithm to the first pixel data set to determine a presence of the structure of interest in the tissue sample; and
(iv) capturing a second pixel data set at a second resolution, the second pixel data set representing an image of the structure of interest, the second resolution providing an increased degree to which closely spaced objects in the image can be distinguished from one another over the first resolution.

89. The system of claim 88, wherein the image capture application further includes:
adjusting an image-capture device to capture a second pixel data set at a second resolution, the second pixel data set representing an image of the structure of interest, the second resolution providing an increased degree to which closely spaced objects in the image can be distinguished from one another over the first resolution.

90. The system of claim 88, wherein the application includes the plurality of structure-identification algorithms.

91. The method of claim 11, wherein the adjusting step further includes changing the light wavelength to provide the second resolution.
